(19) United States
(12) Patent Application Publication
(10) Pub. No.: US 2015/0125489 A1
(43) Pub. Date: May 7, 2015

(54) METHOD FOR THE PREPARATION OF DENDRITIC CELL VACCINES

(71) Applicants: LABORATORIOS DEL DR. ESTEVE, S.A., Barcelona (ES); FUNDACIÓ PRIVADA INSTITUT DE RECERCA DE LA SIDA - CAIXA, Badalona (ES)

(72) Inventors: Felipe García Alcaide, Barcelona (ES); Teresa Gallart, Barcelona (ES); Nùria Climent Vidal, Barcelona (ES); Cristina Gil Roda, Barcelona (ES); Josep María Gatell Artigas, Barcelona (ES)

(21) Appl. No.: 14/382,398

(22) PCT Filed: Mar. 1, 2013
(86) PCT No.: PCT/EP2013/054121

§ 371 Date: Sep. 2, 2014

(30) Foreign Application Priority Data
Mar. 2, 2012 (EP) 12382078.9

(51) Int. Cl. C12N 5/0784; C12N 7/00; A61K 39/21

(52) U.S. Cl. CPC C12N 5/0639; A61K 39/21; C12N 7/00; A61K 2039/5154
USPC 424/208.1; 435/375; 435/325

(57) ABSTRACT

The present invention relates to a process for obtaining an antigen-loaded dendritic cell showing higher viability and migratory capacity towards lymphatic nodes. The invention also relates to vaccines containing said dendritic cells as well as to the use thereof for the treatment of infectious diseases, especially AIDS.

### FIELD OF THE INVENTION

The present invention relates to a process for obtaining an antigen-loaded dendritic cell showing higher viability and migratory capacity towards lymphatic nodes. The invention also relates to vaccines containing said dendritic cells as well as to the use thereof for the treatment of infectious diseases, and particularly, the human immunodeficiency virus (HIV).

### BACKGROUND OF THE INVENTION

Although combined antiretroviral therapy (cART) is effective in suppressing HIV-1 replication and allowing the reconstitution of CD4 T cell counts, it does not eradicate HIV-1. In addition, cART does not restore HIV-1 specific T cell immune responses. In fact, HIV-1 replication rapidly rebounds to similar or even higher pre-treatment levels. Consequently, HIV subjects are compelled to receive cART for life, a particularly burdensome option, concerning compliance, the risk of developing antiviral resistance, price and side effects, including serious metabolic abnormalities, such as fat redistribution syndromes. See Martinez E, et al., Lancet 2001; 357:592-598.

There is evidence that a strong and specific CD4+ helper T cell response against HIV-1 is crucial to achieve a sustained, effective and specific CD8+ cytotoxic T lymphocyte (CTL) response capable of controlling HIV-1 replication in macaques and humans. These findings are consistent with recent data on chronic viral infections in a mouse model. Although HIV-1 specific CD8+ T cells and CD4+ T cells secreting interferon gamma (IFN-γ) can be found in most HIV-1 infected individuals, the CD4+ T cell proliferative response is absent, while the cytolytic activity of CD8 T cells is defective. Some data suggest that the antigen presenting cell (APC) functions of dendritic cells (DCs) are also impaired in HIV-1-infected subjects and this could contribute to dysfunction in HIV-1 specific helper and CTL responses.

Therapeutic immunization has been proposed as an approach to limit the need for continuous lifelong cART. Myeloid dendritic cells are the most potent professional APCs with the unique ability to induce primary and secondary immune responses to both CD4+ and CD8+ T cells. In vivo and in vitro experimental data have shown that DCs are able to engulf exogenous soluble proteins, tumor cell lysates, inactivated viruses and apoptotic virus-infected cells, process these materials, and present derived antigenic peptides. In addition to presenting antigens via the MHC-class II pathway to helper CD4+ T cells (Th), DCs can also present antigens in the MHC-class I pathway to cytotoxic CD8+ T lymphocytes (CTL), a phenomenon known as “cross-priming” or “cross-presentation”. See Banchereau, Nature 392 (1998): 245-252 and Annu. Rev. Immunol. (2000) 18;767-811, and Larsson M, et al., Curr. Top. Microbiol. Immunol. 2003; 276:261-275.

Autologous myeloid DCs, such as monocyte-derived DCs (MDDCs), pulsed ex vivo with a variety of inactivated pathogens and tumor antigens, have been shown to induce a potent protective immunity in experimental murine models of human infections and tumors. Some studies in animals suggest that DCs loaded with HIV-1 viral lysate, envelope glycoproteins, inactivated virus or nanoparticles mount a potent immune response against HIV-1.

Several DC-based vaccination clinical trials for HIV-1 infection in humans have been published to date. See Kundu S, et al., AIDS Res. Hum. Retroviruses 1998; 14:551-560, Lu W, et al., Nat. Med. 2004; 10:1359-1365, Garcia F, et al., J. Infect. Dis. 2005; 195:1680-1685, Ide F, et al., J. Med. Virol. 2006; 78:711-718, Connolly N, et al., Clin. Vaccine Immunol. 2008; 15:284-292, Gandhi R, et al., Vaccine 2009; 27:6088-6094, Kloverpris H, et al., AIDS 2009; 23:1329-1340, Routy J, et al., Clin. Immunol. 2010; 134:140-147, and Garcia F, et al., J. Infect. Dis. 2011; 203:473-478. There are also some ongoing clinical trials using DCs as a therapeutic vaccine. Regretfully, the results reported have been uneven probably due to the wide variability in the immunogen selected, the methods of inactivation, the culture and pulsing conditions of the DCs and the vaccine administration regime. There is still a need in the art for HIV-1 vaccines based on dendritic cells prepared under standardized processes that will enhance their safety and efficacy profiles.

### SUMMARY OF THE INVENTION

In a first aspect, the invention relates to an in vitro method for obtaining an antigen-loaded dendritic cell which comprises

contacting immature dendritic cells with an immunogen comprising said antigen under conditions adequate for maturation of said immature dendritic cells and under conditions which prevent the adhesion of the cells to the substrate.

In another aspect, the invention relates to an antigen-pulsed dendritic cell obtainable by a method according to the invention.

In another aspect, the invention relates to a dendritic cell vaccine comprising the antigen-pulsed dendritic cell according to the invention.

In another aspect, the invention relates to a dendritic cell vaccine according to the invention for use in medicine.

In yet another aspect, the invention relates to a dendritic cell vaccine according to the invention wherein the immunogen is an HIV immunogen for use in the treatment or prevention of a HIV-infection or of a disease associated with a HIV infection.

### BRIEF DESCRIPTION OF THE FIGURES

FIG. 1. CD80 and CD83 expression levels in MDDC isolated from treated HIV subjects.

FIG. 2. Viability and number of MDDCs isolated from treated HIV subjects.

FIG. 3. Chemotactic properties of MDDCs isolated from treated HIV subjects.

FIG. 4. T cell specific HIV response of the MDDCs isolated from treated HIV subjects.

FIG. 5. Change in pVL from baseline (before any antiretroviral therapy) after immunizations and second interruption of antiretroviral therapy. (A) Median values. (B) Individual values. Numbers at the bottom represent patients at risk. P values of Mann-Whitney U test are shown at weeks 0, 8, 12, 24, 36, and 48. P value of area under the curve (AUC) is also shown (C) HIV viral load in treated HIV subjects at 8, 12, 24, 36 and 48 weeks after vaccination. Values for weeks (−4, −2, 0, 8, 12, 24, 36 and 48) are shown for ARMI, ARMII and ARMIII.

FIG. 6. Scheme of clinical trial design. Thirty-six antiretroviral-treated chronic HIV-1-infected patients were randomized to receive three immunizations with at least 107 MD-DCs pulsed with heat-inactivated autologous virus (109 copies per dose). Patients were followed up to 48 weeks after the first immunization. Week 0 was considered the day of second interruption of cART (2^{nd} STOP). The DC-HIV-1 group received immunizations at weeks −4, −2, and 0 in 12 patients and at weeks 0, 2, and 4 in 12 patients. These two different schedules were selected to assess whether cART could have any influence in the response to immunizations. Because a significant difference in pVL changes or HIV-specific T cell responses between these two schedules was not observed, immunized patients have been analyzed as a single group. DC-control group patients received injection at weeks −4, −2, and 0.

### DETAILED DESCRIPTION OF THE INVENTION

The present invention discloses a new and advantageous method for pulsing monocyte-derived dendritic cells (MDDC) cells with a lysate of essentially inactivated human immunodeficiency virus (HIV). In particular, the pulsed MDDCs of the invention are cultured in an ultra low attachment flasks with a maturation cocktail composed of IL-1-β, IL-6, TNF-α, and PGE₂. The combination of the lack of cell adherence and the culture medium increases significantly the expression of maturation markers in MDDCs (i.e. CD80, CD83), as well as the overall quantity and viability of the pulsed MDDCs. The process of the invention also increases the ex vivo migration capacity of MDDCs and improves their presentation of the HIV-1 antigen to T cells, thus favoring a higher specific immune response against HIV-1. The MDDCs thus pulsed can used as a dendritic cell vaccine for use in human health.

1. Definitions of General Terms and Expressions

The term “AIDS”, as used herein, refers to the symptomatic phase of HIV infection, and includes both Acquired Immune Deficiency Syndrome (commonly known as AIDS) and “ARC,” or AIDS-Related Complex. See Adler M, et al., Brit. Med. J. 1987; 294: 1145-1147. The immunological and clinical manifestations of AIDS are well known in the art and include, for example, opportunistic infections and cancers resulting from immune deficiency.

The term “adjuvant” refers to a substance which, when added to an immunogenic agent, nonspecifically enhances or potentiates an immune response to the agent in a recipient host upon exposure to the mixture.

The term “agonist of the IL-1 receptor”, as used herein, refers to a cytokine that acts as an agonist of the interleukin-1 receptor (IL-1R). Agonists of the IL-1 receptor include, without limitation, IL-1α and IL-1β.

The term “aldrithiol-2” or “2,2′-dithiodipyridine”, as used herein, refers to a chemical agent also known as “aldrithiol” or AT-2, that is a mild oxidizing reagent that eliminates the infectivity of HIV by preferential covalent modification of the free sulfhydryl groups of the cysteines of internal virion proteins, in particular, the nucleocapsid proteins. The AT-2 inactivated virions are non-infectious but able to interact with cell surface receptors and with dendritic cells.

The term “amotosalen” as used herein, refers to a synthetic psoralen compound that intercalates into the helical regions of DNA and RNA reversibly. Since amotosalen is a photoactive compound, it is necessary to use a long-wavelength ultraviolet (UVA) illumination to photochemically treat HIV. Upon illumination with UVA light at 320 to 400 nm, amotosalen forms covalent bonds with pyrimidine bases in nucleic acid. The genomes of pathogens and leukocytes cross-linked in this manner can no longer function or replicate.

The term “antigen”, as used herein, refers to any molecule or molecular fragment that, when introduced into the body, induces a specific immune response (i.e. humoral or cellular) by the immune system. Antigens have the ability to be bound at the antigen-binding site of an antibody. Antigens are usually proteins or polysaccharides. Antigens suitable for the present invention are parts of bacteria, viruses, parasites and other microorganisms such as coats, capsules, cell walls, flagella, fimbriae and toxins. Examples of antigens according to the present invention include antigens from picornavirus, coronavirus, togavirus, flavirvirus, rhabdovirus, paramyxovirus, orthomyxovirus, bunyavirus, arenavirus, reovirus, retrovirus, papilomavirus, parvovirus, herpesvirus, poxvirus, hepadnavirus, and spongiform virus families; or from other pathogens such as trypanosomes, tapeworms, roundworms, helminthes or malaria. Examples of suitable viral antigens are, without limitation: retroviral antigens from the human immunodeficiency virus (HIV) including gene products of the gag, pol, env and nef genes, and other HIV components; hepatitis viral antigens, such as the S, M, and L proteins of hepatitis B virus, the pre-S antigen of hepatitis B virus, and other hepatitis (e.g. hepatitis A, B, and C, viral components such as hepatitis C viral RNA); influenza viral antigens, such as hemagglutinin and neuraminidase and other influenza viral components; measles viral antigens, such as the measles virus fusion protein and other measles virus components; rubella viral antigens, such as proteins E1 and E2 and other rubella virus components; rotaviral antigens, such as VP7sc and other rotaviral components; cytomegaloviral antigens, such as envelope glycoprotein B and other cytomegaloviral antigen components; respiratory syncytial viral antigens, such as the RSV fusion protein, the M2 protein and other respiratory syncytial viral antigen components; herpes simplex viral antigens, such as immediate early proteins, glycoprotein D, and other herpes simplex viral antigen components; varicella zoster viral antigens, such as gpl, gpII, and other varicella zoster viral antigen components; Japanese encephalitis viral antigens, such as proteins E, M-E, M-E-NS1, NS1, NS1-NS2A, 80 percent E, and other Japanese encephalitis viral antigen components; rabies viral antigens, such as rabies glycoprotein, rabies nucleoprotein and other rabies viral antigen components. See Fields B, Knipe D, Eds., “Fundamental Virology”, 2nd Ed. (Raven Press, New York, N.Y., US, 1991) for additional examples of viral antigens.

The term “antigen-loaded antigen-presenting cell”, as used herein, refers to a dendritic cell that have captured an antigen and processed it for presentation to CD4 T helper cells and CD8 cytotoxic T lymphocytes in association with HLA-class II and HLA-class I molecules, respectively.

The term “antiretroviral therapy” or “ART”, as used herein, refers to the administration of one or more antiretroviral drugs to inhibit the replication of HIV. Typically, ART involves the administration of at least one antiretroviral agent (or, commonly, a cocktail of antiretrovirals) such as nucleoside reverse transcriptase inhibitor (e.g. zidovudine, AZT, lamivudine (3TC) and abacavir), non-nucleoside reverse transcriptase inhibitor (e.g. nevirapine and efavirenz), and protease inhibitor (e.g. indinavir, ritonavir and lopinavir). The term Highly Active Antiretroviral Therapy (“HAART”) refers to treatment regimens designed to aggressively suppress viral replication and progress of HIV disease, usually consisting of three or more different drugs, such as for example, two nucleoside reverse transcriptase inhibitors and a protease inhibitor.

The term “autologous”, as used herein, means that the donor and recipient of the HIV-1 viral particle and the dendritic cell is the same subject.

The term “cell”, as used herein, is equivalent to “host cell” and is intended to refer to a cell into which a viral genome, a vector or a HIV-1 viral particle of the invention has been introduced. It should be understood that such terms refer not only to the particular subject cell but to the progeny, or potential progeny, of such a cell. Because certain modifications may occur in succeeding generations due to either mutation or environmental influences, such progeny may not, in fact, be identical to the parent cell, but can be still included within the scope of the term as used herein.

The term “comprising” or “comprises”, as used herein, discloses also “consisting of” according to the generally accepted patent practice.

The expression “conditions adequate for maturation”, as used herein, refers to culturing an immature dendritic cell under conditions suitable to achieve the maturation of said cell. Suitable conditions for maturation are well-known by the skilled in the art. Mature dendritic cells can be prepared (i.e. matured) by contacting the immature dendritic cells with effective amounts or concentration of a dendritic cell maturation agent. Dendritic cell maturation agents can include, for example, BCG, IFN-γ, LPS, monophosphoryl lipid A (MPL), eritoran (CAS number 185955-34-4), TNF-α and their analogs. Effective amounts of BCG typically range from about 10⁵ to 10⁷ cfu per milliliter of tissue culture media. Effective amounts of IFN-γ typically range from about 100-1000 U per milliliter of tissue culture media. Bacillus Calmette-Guerin (BCG) is an avirulent strain of M. Bovis. As used herein, BCG refers to whole BCG as well as cell wall constituents, BCG-derived lipoarabidomannans, and other BCG components that are associated with induction of a type 2 immune response. BCG is optionally inactivated, such as heat-inactivated BCG, or formalin-treated BCG. The immature DCs are typically contacted with effective amounts of BCG and IFN-γ for about one hour to about 48 hours. Suitable culture media include AIM-V®, RPMI 1640, DMEM, or X-VIVO 15™. The tissue culture media can be supplemented with amino acids, vitamins, cytokines (e.g. GM-CSF), or divalent cations, to promote maturation of the cells. Typically, about 500 units/mL of GM-CSF is used.

The expression “conditions adequate for processing of the immunogen and presentation by the antigen-presenting cell”, as used herein, refers to the incubation of the dendritic cell in a suitable medium to allow the capture of the immunogen and the processing and presentation of said immunogen to other cells of the immune system.

The term “contacting”, as used herein, refers to the incubation of an immature dendritic cell in the presence of the immunogen destined for loading into the dendritic cell. Immature DCs are capable of capturing and internalizing said immunogens thus becoming an antigen-loaded dendritic cell (also named antigen-pulsed dendritic cell). Antigen capture by immature DCs is mediated by macropinocytosis, receptor-mediated antigen capture and engulfment of apoptotic bodies. Preferably, said incubation is performed at 37° C. for 6 hours. The success of the antigen-loading step or pulse can be assayed by washing the pulsed dendritic cells to remove uncaptured immunogens and lysing said pulsed dendritic cells to measure the intracellular antigen content by an ELISA assay. For example, when the immunogen is a HIV viral particle, the intracellular content in p24^{Gag} antigen, present on the capsid surface of the viral particles, can be assayed.

The term “dendritic cell”, as used herein, refers to any member of a diverse population of morphologically similar cell types found in lymphoid or non-lymphoid tissues. Dendritic cells are a class of “professional” antigen presenting cells, and have a high capacity for sensitizing HLA-restricted T cells. Specifically, the dendritic cells include, for example, plasmacytoid dendritic cells, myeloid dendritic cells (generally used dendritic cells, including immature and mature dendritic cells). Langerhans cells (myeloid dendritic cells important as antigen-presenting cells in the skin), interdigitating cells (distributed in the lymph nodes and spleen T cell region, and believed to function in antigen presentation to T cells). All these DC populations are derived from bone marrow hematopoietic cells. Dendritic cells also include follicular dendritic cells, which are important as antigen-presenting cells for B cells, but who are not derived from bone marrow hematopoietic cells. Dendritic cells may be recognized by function, or by phenotype, particularly by cell surface phenotype. These cells are characterized by their distinctive morphology (having veil-like projections on the cell surface), intermediate to high levels of surface HLA-class II expression and ability to present antigen to T cells, particularly to naive T cells. See Steinman R, et al., Ann. Rev. Immunol. 1991; 9:271-196. The cell surface of dendritic cells is characterized by the expression of the cell surface markers CD1a+, CD4+, CD86+, or HLA-DR+.

The term “dendritic cell maturation agent”, as used herein, refers to a compound capable of producing the maturation of the dendritic cell when the dendritic cell is incubated with said compound.

The term “dendritic cell precursor”, as used herein, refers to any cell capable of differentiating into an immature dendritic cell in the presence of an appropriate cytokine (i.e. G-CSF, GM-CSF, TNF-α, IL-4, IL-13, SCF (c-kit ligand), Flt-3 ligand, or a combination thereof). Examples of dendritic precursor cells include, but are not limited to, myeloid dendritic precursor cells, lymphoid dendritic precursor cells, plasmacytoid dendritic precursor cells and, particularly, monocytes. Phenotypic surface markers expressed by various subsets of dendritic precursor cells are well known in the art and may be used for the purpose of identification, for example, by flow cytometry or using immunohistochemical techniques.

The term “dendritic cell vaccine”, as used herein, refers to a vaccine comprising dendritic cells which are loaded with the antigens against which an immune reaction is desired.

The expression “disease associated with a HIV infection”, as used herein, includes a state in which the subject has developed AIDS, but also includes a state in which the subject infected with HIV has not shown any sign or symptom of the disease.

The expression “disease which requires an immune response against the antigen which is loaded in the antigen-presenting cell”, as used herein, refers to any disease susceptible of being prevented or treated with the administration of an antigen. Suitable diseases include, without limitation, infectious diseases (e.g. HIV) and cancer.

The term “disulfiram”, as used herein, refers to a chemical agent also known as Antabuse® or tetraethylthiuram disulfide, which is an FDA-approved drug that is widely used for the treatment of alcoholism. Said compound also promote metal ejection from the HIV nucleocapsid protein zinc finger domains.

The term “GM-CSF” as used herein refers to granulocyte macrophage colony stimulating factor or granulocyte macrophage colony stimulation factor from any species or source and includes the full-length protein as well as fragments or portions of the protein mouse GM-CSF (GenBank NM 009969) and human GM-CSF (GenBank BC108724). In one embodiment, the GM-CSF is from human or mouse. In another embodiment, the GM-CSF protein lacks the last 10 carboxy terminal amino acid sequences as compared to full length GM-CSF. The term “GM-CSF fragment” as used herein means a portion of the GM-CSF peptide that contains at least 10%, 20%, 30%, 40%, 50%, 60%, 70%, 80%, 90%, 95%, or more of the entire length of the GM-CSF polypeptide that is capable of stimulating stem cells to produce granulocytes (neutrophils, eosinophils, and basophils) and monocytes.

The term “gp130 utilizing cytokine”, as used herein, refer to a cytokine that signal through receptors containing gp130. The signal-transducing component glycoprotein 130 (gp130), also called CD130, is a transmembrane protein that forms one subunit of type I cytokine receptors within the IL-6 receptor family. The gp130 utilizing cytokines (also known as IL-6-like cytokines) useful in the present invention include, interleukin 6 (IL-6), interleukin 11 (IL-11), interleukin 27 (IL-27), ciliary neurotrophic factor (CNTF), cardiotrophin-1 (CT-1), cardiotrophin-like cytokine (CLC), leukemia inhibitory factor (LIF), oncostatin M (OSM) and Kaposi's sarcoma-associated herpesvirus interleukin 6 like protein (KSHV-IL6).

The term “HIV immunogen”, as used herein, refers to a protein or peptide antigen derived from HIV that is capable of generating an immune response in a subject and also refers to a HIV viral particle, being said particle a whole viral particle or a viral particle lacking one or more viral components but retaining the ability to generate an immune response. HIV immunogens for use according to the present invention may be selected from any HIV isolate (e.g. any primary or cultured HIV-1, HIV-2, or HIV-3 isolate, strain, or Glade). HIV isolates are now classified into discrete genetic subtypes. HIV-1 is known to comprise at least ten subtypes (A1, A2, A3, A4, B, C, D, E, PL F2, G, H, J and K). See Taylor B, et al., New Engl. J. Med 2008; 359(18):1965-1966. HIV-2 is known to include at least five subtypes (A, B, C, D, and E). Subtype B has been associated with the HIV epidemic in homosexual men and intravenous drug users worldwide. Most HIV-1 immunogens, laboratory adapted isolates, reagents and mapped epitopes belong to subtype B. In sub-Saharan Africa, India, and China, areas where the incidence of new HIV infections is high, HIV-1 subtype B accounts for only a small minority of infections, and subtype HIV-1 C appears to be the most common infecting subtype. Thus, in certain embodiments, it may be preferable to select immunogens from particular subtypes (e.g. HIV-1 subtypes B or C). It may be desirable to include immunogens from multiple HIV subtypes (e.g. HIV-1 subtypes B and C, HIV-2 subtypes A and B, or a combination of HIV-1, HIV-2, or HIV-3 subtypes) in a single immunological composition.

The term “HIV-1 viral particle”, as used herein, refers to a roughly spherical structure with a diameter of about 120 nm composed of two copies of positive single-stranded RNA that encodes the virus nine genes enclosed by a conical capsid composed of 2,000 copies of the viral protein p24. The single-stranded RNA is tightly bound to nucleocapsid proteins, p7, and enzymes needed for the development of the virion such as reverse transcriptase, proteases, ribonuclease and integrase. A matrix composed of the viral protein p17 surrounds the capsid ensuring the integrity of the virion particle. This is, in turn, surrounded by the viral envelope that is composed of two layers of fatty molecules called phospholipids taken from the membrane of a human cell when a newly formed virus particle buds from the cell. Embedded in the viral envelope are proteins from the host cell and about 70 copies of a complex HIV protein that protrudes through the surface of the virus particle. This protein, known as Env, consists of a cap made of three molecules called glycoprotein (gp) 120, and a stem consisting of three gp41 molecules that anchor the structure into the viral envelope. This glycoprotein complex enables the virus to attach to and fuse with target cells to initiate the infectious cycle.

The term “human immunodeficiency virus” or “HIV”, as used herein is meant to include HIV-1 and HIV-2. “HIV-1” means the human immunodeficiency virus type-1. HIV-1 includes, but is not limited to, extracellular virus particles and HIV-1 forms associated with HIV-1 infected cells. “HIV-2” means the human immunodeficiency virus type-2. HIV-2 includes, but is not limited to, extracellular virus particles and HIV-2 forms associated with HIV-2 infected cells. Preferably, HIV is HIV-1.

The term “immature dendritic cell”, as used herein, refers to a dendritic cell having significantly low T cell-activating ability as compared with a dendritic cell in a matured state. Specifically, the immature dendritic cells may have an antigen-presenting ability that is lower than ½, preferably lower than ¼ of that of dendritic cells which maturation had been induced by adding LPS (1 μg/mL) and culturing for two days. The antigen-presenting ability can be quantified, for example, using the allo T cell-activating ability (mixed lymphocyte test: allo T cells and dendritic cells are co-cultured at a T cell:dendritic cell ratio of 1:10, or preferably at varied ratios; 3H-thymidine is added 8 hours before terminating cultivation, and the T cell growth capacity is assessed based on the amount of 3H-thymidine incorporated into the DNA of the T cells. See Jonuleit H, et al., Gene Ther. 2000; 7:249-254. Alternatively, it can be assessed by testing the ability to induce specific cytotoxic T cells (CTLs) using a peptide, in which a known class I-restricted peptide of a certain antigen is added to dendritic cells; the dendritic cells are co-cultured with T cells obtained from peripheral blood of the same healthy donor from whom the dendritic cells had been collected (with 25 U/mL or preferably 100 U/mL of IL-2 on day 3 or later). The T cells are preferably stimulated with dendritic cells three times during 21 days, more preferably stimulated with dendritic cells twice during 14 days. The resulting effector cells are co-cultured for four hours with 51Cr-labeled target cells (peptide-restricted class I positive tumor cells) at a ratio of 100:1 to 2.5:1 (100:1, 50:1, 25:1, 20:1, 12.5:1, 10:1, 5:1, or 2.5:1), preferably at a ratio of 10:1; and 51Cr released from the target cells is quantified. See Hristov G, et al., Arch. Dermatol. Res. 2000; 292:325-332. Furthermore, the immature dendritic cells preferably have phagocytic ability for antigens, and more preferably show low (for example, significantly low as compared to mature DCs induced by LPS as described above) or negative expression of receptors that induce the co-stimulation for T cell activation. Immature dendritic cells express surface markers that can be used to identify such cells by flow citometry or immunohistochemical staining.

The term “immunogen”, as used herein, refers to an antigen capable of provoking an adaptative immune response if injected by itself. All immunogens are also antigens but not all antigens are immunogens.

The term “immunogenic composition”, as used herein, refers to a composition that elicits an immune response in a subject that produces antibodies or cell-mediated immune responses against a specific immunogen. Immunogenic compositions can be prepared, for instance, as injectables such as liquid solutions, suspensions, and emulsions. The term “antigenic composition” refers to a composition that can be recognized by a host immune system. For example, an antigenic composition contains epitopes that can be recognized by humoral or cellular components of a host immune system.

As used herein, the term “inactivated HIV virus” refers to an intact, inactivated HIV virus. An inactivated HIV refers to a virus that cannot infect or replicate. A whole inactivated HIV virus generally maintains native structure of viral antigens to maintain immunogenicity and stimulate immune responses to native virus.

The term “incubation”, as used herein, refers to maintaining the culture of the dendritic cells in a maturation medium during a specific time, preferably during 48 hours, until the immature dendritic cell is transformed in a mature dendritic cell. The term “medium” is maturation substrate comprising a suitable culture media, one or more maturation agents and, optionally, other supplements.

The term “IL-4” as used herein, refers to interleukin-4 of any species, native or recombinant, having the 129 normally occurring amino acid sequence of native human IL-4 (SEQ ID NO: 1), and variants thereof which maintain the ability to promote Th2 cell differentiation, immunoglobulin class switch, and antibody production in B cells. See Lee F, et al., U.S. Pat. No. 5,017,691. IL-4 activity can be measured, for example, by immunological procedures such as ELISA, or EIA.

The term “lysate of essentially inactivated HIV” refers to the solution produced when cells are destroyed that contains HIV virions which have been submitted to an inactivation procedure with a chemical agent in which at least 20%, at least 30%, at least 40%, at least 50, at least 60%, at least 70%, at least 80%, at least 90% or 100% of said virus are inactivated.

The term “mature dendritic cell”, as used herein, is a cell that has significantly strong antigen-presenting ability for T cell or the like as compared with a dendritic cell in the immature state. Specifically, the mature dendritic cells may have an antigen-presenting ability that is half or stronger, preferably equivalent to or stronger than the antigen-presenting ability of dendritic cells in which maturation has been induced by adding LPS (1 μg/mL) and culturing for two days. Mature DC display up-regulated expression of co-stimulatory cell surface molecules and secrete various cytokines. Specifically, mature DCs express higher levels of HLA class I and class II antigens (HLA-A, B, C, HLA-DR) and are generally positive for the expression of CD80, CD83 and CD86 surface markers.

The expression “median tissue culture infective dose” or “TCID50”, as used herein, means the amount of a pathogenic agent that will produce pathological change in 50% of cell cultures inoculated.

The term “medicament”, as used herein, is understood to be a pharmaceutical composition, particularly a vaccine, comprising the immunogenic composition of the invention.

The term “monocytic dendritic cell precursors” or MoDC precursors, as used herein, comprises monocytes that have the GM-CSF receptor on their surface and other myeloid precursor cells that are responsive to GM-CSF. The cells can be obtained from any tissue where they reside, particularly lymphoid tissues such as the spleen, bone marrow, lymph nodes and thymus. Monocytic dendritic cell precursors also can be isolated from the circulatory system. Peripheral blood is a readily accessible source of monocytic dendritic cell precursors. Umbilical cord blood is another source of monocytic dendritic cell precursors.

The term “operably linked”, as used herein, is intended to mean that the nucleotide sequence of interest is linked to the regulatory sequence(s) in a manner that allows for expression of the nucleotide sequence (e.g. in an in vitro transcription/translation system or in a host cell when the vector is introduced into the host cell). See Auer H, Nature Biotechnol. 2006; 24: 41-43.

The terms “pharmaceutically acceptable carrier,” “pharmaceutically acceptable diluent,” “pharmaceutically acceptable excipient”, or “pharmaceutically acceptable vehicle”, used interchangeably herein, refer to a non-toxic solid, semisolid or liquid filler, diluent, encapsulating material or formulation auxiliary of any conventional type. A pharmaceutically acceptable carrier is essentially non-toxic to recipients at the employed dosages and concentrations and is compatible with other ingredients of the formulation. The number and the nature of the pharmaceutically acceptable carriers depend on the desired administration form. The pharmaceutically acceptable carriers are known and may be prepared by methods well known in the art. See Faulí i Trillo C, “Tratado de Farmacia Galénica” (Ed. Luzán 5, S. A., Madrid, ES, 1993) and Gennaro A, Ed., “Remington: The Science and Practice of Pharmacy” 20th ed. (Lippincott Williams & Wilkins, Philadelphia, Pa., US, 2003).

The term “prevention”, as used herein, means the administration of an immunogenic composition of the invention or of a medicament containing it in an initial or early stage of the infection, to avoid the appearance of clinical signs.

The expression “pro-inflammatory cytokine cocktail”, as used herein, refers to a mixture of two or more cytokines that are able to trigger the maturation of immature dendritic cells. Examples of such cytokines are, without limitation, IL-1-β, IL-6, TNF-α, IL-18, IL-11, IL-27, and IFN-α. Suitable pro-inflammatory cytokine cocktails are, without limitation, a cocktail formed by TNF-α and CD40L; a cocktail formed by IFN-α and TNF-α; a cocktail formed by IFN-α and CD40L; a cocktail formed by IFN-α, TNF-α and CD40L; a cocktail formed by TNF-α, IL-1-β_0 and IL-6; a cocktail formed by IL-1β, TNF-α, IFN-α, IFN-γ and poly (I:C); a cocktail formed by IL-1, IL-6, TNF-α, IFN-α, and CD40L.

The term “prostaglandin”, as used herein, refers to a member of a group of lipid compounds that are derived enzymatically from fatty acids and have important functions in the animal body. Every prostaglandin contains 20 carbon atoms, including a 5-carbon ring. Examples of prostaglandins useful in the present invention are, without limitation, prostacyclin I₂ (PGI₂), prostaglandin E₂ (PGE₂) and prostaglandin F_{2α} (PGF_{2α}).

The term “psoralen compound”, as used herein, refers to a compound pertaining to a family of natural products known as furocoumarins that are photoactive compounds. Said compounds intercalate into the DNA and, on exposure to ultraviolet (UVA) radiation, can form covalent interstrand crosslinks with thymines at 5′-TpA sites in the genome, preferentially.

The term “TLR4 ligand” or “toll-like receptor 4 ligand”, as used herein, refers to a ligand of the toll-like receptor 4 (TLR4). TLR4 has also been designated as CD284 (cluster of differentiation 284) and is a member of the Toll-like receptor family, which plays a fundamental role in pathogen recognition and activation of the innate immune system.

The term “TNF superfamily member”, as used herein, refers to a cytokine that pertains to the tumor necrosis factor (TNF) superfamily. The TNF superfamily of cytokines represents a multifunctional group of pro-inflammatory cytokines which activate signaling pathways for cell survival, apoptosis, inflammatory responses and cell differentiation. Examples of TNF superfamily members include, without limitation, tumor necrosis factor alpha (TNF-α), LIGHT, CD40 ligand (CD40L), 4-1BB ligand (4-1BBL), APRIL, CD27 ligand (CD27L), CD30 ligand (CD30L), Fas ligand, glucocorticoid-induced TNFR-related ligand (GITRL), lymphotoxin alpha (LTα), lymphotoxin beta (LTβ), OX40 ligand (OX40L), receptor activator of NF-κB ligand (RANKL), B cell-activating factor of the TNF family (BAFF), TNF-related apoptosis-inducing ligand (TRAIL), TNF-like weak inducer of apoptosis (TWEAK) and VEG1.

The term “treat” or “treatment”, as used herein, refers to the administration of an immunogenic composition of the invention or of a medicament containing it to control the progression of the disease before or after clinical signs have appeared. Control of the disease progression is understood to mean the beneficial or desired clinical results that include, but are not limited to, reduction of the symptoms, reduction of the duration of the disease, stabilization of pathological states (specifically to avoid additional deterioration), delaying the progression of the disease, improving the pathological state and remission (both partial and total). The control of progression of the disease also involves an extension of survival, compared with the expected survival if treatment was not applied. Within the context of the present invention, the terms “treat” and “treatment” refer specifically to preventing or slowing the infection and destruction of healthy CD4+ T cells in a HIV-1 infected subject. It also refers to the prevention and slowing the onset of symptoms of the acquired immunodeficiency disease such as extreme low CD4+ T cell count and repeated infections by opportunistic pathogens such as Mycobacteria spp., Pneumocystis carinii, and Pneumocystis cryptococcus. Beneficial or desired clinical results include, but are not limited to, an increase in absolute naïve CD4+ T cell count (range 10-3520), an increase in the percentage of CD4+ T cell over total circulating immune cells (range 1-50%), and/or an increase in CD4+ T cell count as a percentage of normal CD4+ T cell count in an uninfected subject (range 1-161%). “Treatment” can also mean prolonging survival of the infected subject as compared to expected survival if the subject did not receive any HIV targeted treatment.

The term “vaccine”, as used herein, refers to an immunogenic composition for in vivo administration to a host, which may be a primate, especially a human host, to confer protection against a disease, particularly a viral disease.

The term “vector”, as used herein, denotes a nucleic acid molecule, linear or circular, that comprises the genome encoding all the proteins forming a viral particle (except a part or the complete integrase protein) operably linked to additional segments that provide for its autonomous replication in a host cell of interest. Preferably, the vector is an expression vector, which is defined as a vector, which in addition to the regions of the autonomous replication in a host cell, contains regions operably linked to the genome of the invention and which are capable of enhancing the expression of the products of the genome according to the invention.

The term “viral particle”, as used herein, refers to a whole viral particle and not to a protein subunit or peptide. Viral particles (also known as virions) consist of two or three parts: the genetic material of the virus made from either DNA or RNA; a protein coat that protects these genes; and, in some cases, an envelope of lipids that surrounds the protein coat when they are outside a cell. The shape of the viral particle ranges from simple helical and icosahedral forms to more complex structures, depending on the virus.

2. Method for Obtaining an Antigen-Loaded Antigen Presenting Cell In Vitro

In a first aspect, the invention relates to an in vitro method for obtaining an antigen-loaded dendritic cell (hereinafter referred to as “first method of the invention”) which comprises contacting immature dendritic cells with an immunogen comprising said antigen under conditions adequate for maturation of said immature dendritic cells and under conditions which prevent the adhesion of the cells to the substrate.

The method of the invention comprises contacting immature dendritic cells with an immunogen comprising an antigen under conditions adequate for: a) maturing the antigen presenting cell and b) preventing the adhesion of the cells to the substrate.

In a preferred embodiment, the immunogen is a viral particle, preferably, an HIV viral particle, more preferably, an HIV-1 viral particle. The viral particle may contain several antigens.

The HIV-1 virus exhibits an unusually high degree of genetic variability throughout its genome. Sequence comparisons have identified three genetic groups of HIV-1, designated M, O, and N. The existence of a fourth group, “P”, has been hypothesized based on a virus isolated in 2009. Group M is further divided into phylogenically related major genetic subtypes (or clades), designated A, B, C, D, E, F, G, H, J and K. Co-infection with distinct subtypes gives rise to circulating recombinant forms (CRFs). Together with circulating inter-subtype recombinant forms (CRFs), group M comprises the majority of HIV-1 variants in the world today. The HIV-1 virus of the present invention may represent any of the genetic groups or genetic subtypes capable of infecting a human being, and also includes circulating recombinant forms, laboratory strains and primary isolates. Thus, in a preferred embodiment the immunogen is an HIV immunogen.

Suitable HIV immunogens include the HIV envelope (env; e.g. NCBI Ref. Seq. NPJ357856), gag (e.g. p6, p7, p17, p24, GenBank AAD39400.1), pol encoded protease (e.g. UniProt P03366), nef (e.g. GenBank CAA41585.1, Shugars D, et al., J. Virol. 1993; 67(8):4639-4650), as well as variants, derivatives, and fusion proteins thereof. See Gómez C, et al., Vaccine 2007; 25:1969-1992. Suitable strains and combinations may be selected by the skilled artisan as desired.

The HIV immunogen of the invention is capable of eliciting an immune response. Particularly, “immune response” refers to a CD4+ T cell or CD8+ T cell mediated immune response to HIV infection. An immune response to HIV may be determined by measuring, for example, viral load, T cell proliferation, T cell survival, cytokine secretion by T cells, or an increase in the production of antigen-specific antibodies (e.g. antibody concentration).

The first step of the method is carried out under conditions adequate for maturation of said antigen presenting cell. In a preferred embodiment, the conditions adequate for maturation of the immature dendritic cell comprise the contacting with a combination of GM-CSF and IL-4.

GM-CSF may be used in concentrations of 100 to 1500 IU/mL preferably between 300 to 1300 IU/mL, more preferably between 500 and 1200 IU/mL, such as for example 700 to 1100 IU/mL and most preferably at about 1000 IU/mL. Either purified GM-CSF or recombinant GM-CSF, for example, recombinant human GM-CSF (R&D Systems, Inc., Minneapolis, Minn., US) or sargramostim (Leukine®, Bayer Healthcare Pharmaceuticals, Inc., Wayne, N.J., US) can be used in the methods described herein.

IL-4 may be used in concentrations of 100 to 1500 IU/mL, preferably, between 300 to 1300 IU/mL, more preferably, between 500 and 1200 IU/mL, such as for example 700 to 1100 IU/mL, and most preferably, at about 1000 IU/mL.

In a preferred embodiment, both cytokines (GM-CSF and IL-4) are used at concentrations of 1000 IU/mL.

In an attempt to recreate a physiological environment for DC maturation, some balanced cocktails of maturation agents can be used. Thus, in another preferred embodiment, the cell maturation agent is a pro-inflammatory cytokine cocktail. In a preferred embodiment, the pro-inflammatory cytokine cocktail comprises at least an agonist of the IL-1 receptor, a gp130 utilizing cytokine and a TNF superfamily member. Said cytokine cocktail can include other compounds.

In a preferred embodiment, the IL-1 receptor agonist is IL-1β. Preferably, the effective IL-1β concentration is 300 U/mL. In another preferred embodiment, the gp130 utilizing cytokine is IL-6. Preferably, the effective IL-6 concentration is 1000 U/mL of IL-6. In another preferred embodiment, the TNF superfamily member is TNF-α. Preferably, the effective TNF-α concentration is 1000 U/mL.

The most frequently used cocktail contains TNF-α, IL-1β and IL-6. Thus, in a preferred embodiment the pro-inflammatory cytokine cocktail comprises a mixture of IL-1β, IL-6 and TNF-α. More preferably, the composition of the medium is 300 U/mL of IL-1β, 1000 U/mL of TNF-α and 1000 U/mL of IL-6.

It has been disclosed that the addition of a prostaglandin to the pro-inflammatory cytokine cocktail improves the yield, maturation, migratory and immunostimulatory capacity of the DC generated. See Jonuleit H, et al., Eur. J. Immunol. 1997; 27: 3135-3142. Thus, in a preferred embodiment the pro-inflammatory cytokine cocktail further comprises a prostaglandin. More preferably, the prostaglandin utilized is prostaglandin E₂ (PGE₂). Preferably, the effective PGE₂ concentration is 1 μg/mL. More preferably, the composition of the medium is 300 IU/mL of IL-1β, 1000 IU/mL of TNF-α, 1000 IU/mL of IL-6 and 1 μg/mL of PGE₂.

In another embodiment, the contacting step involves a first step wherein the cells are contacted with a combination of GM-CSF and IL-4 and a second step wherein the cells are contacted with a pro-inflammatory cytokine cocktail as defined above. In another embodiment, the contacting step involves a first step wherein the cells are contacted with a combination of GM-CSF and IL-4 and a second step wherein the cells are contacted with a combination of GM-CSF and IL-4 and a pro-inflammatory cytokine cocktail as defined above.

The first method of the invention is also carried out under conditions which prevent the adhesion of the cells to the substrate. The cells are considered as being non-adherent if the cells can be collected with the supernatant from the culture recipient after the application of soft mechanical forces (e.g. slight tapping of the flask) to detach weakly-adhering cells. In a preferred embodiment, the cells are prevented from adhering to the substrate using a low adherence substrate. These substrates are widely available and are usually formed by hydrogels which are hydrophilic and neutrally charged, thus preventing the attachment of cells via the interaction with negatively or positively charged surface proteins or hydrophobic interactions. A substrate is considered as low adherence wherein it results in the attachment of a monocyte cell population which is at least 10%, at least 20%, at least 30%, at least 40%, at least 50%, at least 60%, at least 70%, at least 80%, at least 90% or less than the attachment to an adherent substrate (e.g. a polystyrene substrate). Suitable assays for determining whether a surface is low-adherent are known in the art. See Shen M, et al., J. Biomed. Mater. Res. 2001; 57:336-345.

The method of the invention is carried out by using immature dendritic cells which develop to mature dendritic cells when contacted with a maturation composition.

Immature dendritic cells can be obtained from a population of dendritic cell precursors. Preferably, the dendritic cell precursor is a cell that can differentiate into an immature dendritic cell in four weeks or less, more preferably, in 20 days or less, even more preferably, in 18 days or less, and still more preferably, in 16 days or less. In a preferred embodiment, the dendritic cell precursor differentiates into an immature dendritic cell in the presence of GM-CSF and IL-4 in less than seven days, and more preferably, in five days.

In a preferred embodiment, the population of dendritic precursor cells is a population of monocytic dendritic cell precursors. More preferably, the monocytic dendritic cell precursors are derived from peripheral blood mononuclear cells (PBMCs). The PBMCs can be obtained either from whole blood diluted 1:1 with buffered saline or from leukocyte concentrates (“buffy coat” fractions, MSKCC Blood Bank) by standard centrifugation over Ficoll-Paque PLUS (endotoxin-free, catalogue number 17-1440-03, Amersham Pharmacia Biotech AB, Uppsala, SE). MoDC precursors are tissue culture plastic-adherent (catalogue number. 35-3003, Falcon, Becton-Dickinson Labware Inc., Franklin Lakes, N.J., US) PBMCs, and can be cultured in complete RPMI 1640 plus 1% normal human serum (NHS) (or 10% fetal bovine serum) in the presence of GM-CSF (1000 IU/mL) and IL-4 (500 IU/mL) with replacement every 2 days as described. See Thurner B, et al., J. Immunol. Meth. 1999; 223:1-15 and Ratzinger G, et al., J. Immunol. 2004; 173:2780-2791.

Purified monocyte populations can be isolated from PBMCs with CD14⁺ antibodies prior to the culture to obtain immature dendritic cells. Monocytes are usually identified in stained smears by their large bilobate nucleus. In addition to the expression of CD14, monocytes express also, among others, one or more of the following surface markers: 125I-WVH-1, 63D3, adipophilin, CB12, CD1 Ia, CD1 Ib, CD15, CD54, Cd163, cytidine deaminase, and FIt-I. See Feyle D, et al., Eur. J. Biochem. 1985; 147:409-419, Malavasi F, et al., Cell Immunol. 1986; 97(2):276-285, Rupert J, et al., Immunobiol. 1991; 182(5):449-464; Ziegler-Heitbrock H, J. Leukoc. Biol. 2000; 67:603-606, and Pilling D, et al., PLoS One 2009; 4(10):e-7475.

In general, monocytic dendritic cell precursors may be identified by the expression of markers such as CD13 and CD33. Myeloid dendritic precursors may differentiate into dendritic cells via CD14 or CD1a pathways. Accordingly, a dendritic precursor cell of the invention may be a CD14+ CD1a− dendritic precursor cell or a CD14−CD1a+ dendritic precursor cell. In certain embodiments of the invention, a myeloid dendritic precursor cell may be characterized by the expression of SCA-1, c-kit, CD34, CD16, and CD14 markers. In a preferred embodiment, the myeloid dendritic precursor cell is a CD14+ monocyte. The CD14+ monocyte may also express the GM-CSF receptor.

The immature dendritic cells used as starting material for the first method of the invention can be autologous to the subject to be treated. In other embodiments, the immature dendritic cells used as starting material for the methods of the invention are heterologous dendritic cells. For example, if graft-versus-host disease is to be treated, the immature dendritic cells that are being used as starting material are dendritic cells that were obtained from the donor. The subject can be, for instance, a mouse, a rat, a dog, a chicken, a horse, a goat, a donkey, or a primate. Most preferably, the subject is a human. In a preferred embodiment, the immature dendritic cell is a monocyte-derived immature dendritic cell.

The first method of the invention comprises contacting said immature dendritic cells with an immunogen comprising said antigen under conditions adequate for maturation of said antigen presenting cell and under conditions which prevent the adhesion of the cells to the substrate. As a result, an antigen-loaded antigen-presenting cell is obtained.

At the end of the incubation time a mature antigen-loaded dendritic cell is obtained (i.e. a mature dendritic cell carrying the antigen of interest). Maturation of dendritic cells can be monitored by methods known in the art. mDCs surface markers can be detected in assays such as flow cytometry and immunohistochemical staining. The mDCs can also be monitored by cytokine production (e.g. by ELISA, another immune assay, or by use of an oligonucleotide array). The maturation of a dendritic cell can be further confirmed by immunophenotyping. An immature dendritic cell may be distinguished from a mature dendritic cell, for example, based on markers selected from the group consisting of CD80 and CD86. An immature dendritic cell is weakly positive and preferably negative for these markers, while a mature dendritic cell is positive.

When the method of the invention takes place in a culture having a population of immature dendritic cells, conditions adequate for maturation are such where the maturation of at least 50%, at least 60%, at least 70%, at least 80%, at least 90%, or preferably, 100% of immature dendritic cells, is achieved.

In a preferred embodiment, the first method of the invention further comprises recovering the immunogen-pulsed dendritic cells. Said recovery can be carried out by any method known in the art. In a preferred embodiment, the recovery of the immunogen-pulsed dendritic cells is carried out by immunoisolation using antibodies specific for markers of mature dendritic cells such as one or more of the group consisting of CD4, CD8, CD54, CD56, CD66b, and CD86.

In a preferred embodiment, the immunogen to be loaded into a dendritic cell is a viral particle, preferably, a retroviral viral particle. In another preferred embodiment, the immunogen is a lentivirus particle, preferably, an HIV viral particle. More preferably, the immunogen is an HIV-1 viral particle.

HIV-1 virus binds with and subsequently infects human CD4 cells through the use of a co-receptor on the cell surface. Different strains of HIV-1 use different co-receptors to enter human CD4 cells. Thus, HIV-1 virus can be CCR5-tropic when the virus strain only uses the C—C chemokine receptor type 5 (CCR5) co-receptor to infect CD4 cells; CXCR4-tropic when a virus strain only uses the C—X—C chemokine receptor type 4 (CXCR4) co-receptor to infect the CD4 cells; and dual-tropic when the virus strain can use either the CCR5 or CXCR4 co-receptor to infect CD4 cells. See Whitcomb J, et al., Antimicrob. Agents Chemother. 2007; 51(2):566-575. There are available several assays to distinguish between different tropic viruses (e.g. Trofile®, Monogram Biosciences, Inc., San Francisco, Calif., US). In a preferred embodiment, the HIV-1 virus is selected from a CXCR4-tropic virus and a CCR5-tropic virus; preferably, it is a CXCR4-tropic virus.

In another embodiment, the immunogen is an inactivated HIV particle or a lysate of essentially inactivated HIV. The virus or the lysate thereof can be inactivated using conventional means, such as heat, chemical agents and photochemical agents.

An inactivated virus is not detectably infectious in vitro. To quantify the reduction in the infective dose produced by the inactivation process applied and to quantify the residual infective dose that remains in the sample after the inactivation, the inactivated HIV is submitted to an assay. Methods that can be used to this purpose are known in the art. See Agrawal K, et al., PLoS One. 2011; 6(6):e21339. The methods include the use of inactivated supernatnats for infecting permissible cells followed by detection of the newly formed virus. Said detection can be carried out by measuring the number of HIV RNA copies/mL produced by the cells or the amount of HIV p24 antigen/mL of supernatant by the ELISA method. The detection of the production of HIV p24 antigen can be carried out, for instance, by ELISA as described in the experimental part of the present invention.

The inactivation step is carried out for sufficient time so as to result in an decrease in infectivity of the supernatant with respect to a control supernatant (i.e. a supernatant which has not been treated with the inactivating agent or which has been treated under similar conditions with the vehicle in which the inactivating agent is provided) of at least 10%, at least 20%, at least 30%, at least 40%, at least 50%, at least 60%, at least 70%, at least 80%, at least 90% or at least 100%. Suitable methods for assessing HIV inactivation entails, without limitation, taking blood cultures followed by culturing in a T cell media and measuring infectivity. An alternative method is to determine the virus copies that are present in the blood before and after the inactivation attempt or treatment in a periodic fashion (e.g. every 1-7 days).

In one embodiment, the immunogen is a heat-inactivated virus or virus lysate. Viruses, such as HIV-1, may be heat-inactivated by several known protocols in the art. See Harper J, et al., J. Virol. 1978; 26(3):646-659, Einarsson R, et al., Transfusion 1989; 29(2):148- 152, and Gil C, et al., Vaccine 2011; 29(34): 5711-5724.

In another embodiment, the immunogen is chemically-inactivated virus or virus lysate. The inactivation may be attained by incubating the virus with a chemical agent. In a further aspect of the present invention, the mixture of the virus and the chemical agent is irradiated. Preferably, the mixture is irradiated with ultraviolet light until the virus is inactivated.

In a preferred embodiment, the chemical agent is a zinc finger-modifying compound. The term “zinc finger-modifying compound” refers to a compound that covalently modifies the essential zinc fingers in the nucleocapsid protein of HIV virions, thereby inactivating infectivity. The advantage of such a mode of inactivation is that the conformational and functional integrity of proteins on the virion surface is preserved. A number of compounds have been identified that act via a variety of different mechanisms to covalently modify the nucleocapsid zinc fingers, resulting in ejection of the coordinated zinc and loss of infectivity. Despite differences between detailed mechanisms of action for these compounds, the common mechanistic feature involves a preferential chemical attack on the zinc-coordinating cysteine sulfurs in the residues that make up the nucleocapsid protein zinc fingers. See Rossio J, et al., J. Virol. 1998; 72(10):7992-8001).

Suitable zinc finger-modifying compounds for use in the process according to the present invention include, without limitation:

(i) a C-nitroso compound,

(ii) azodicarbonamide,

(iii) a disulphide having the structure R—S—S—R,

(iv) a maleimide having the structure

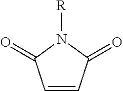

(v) an alpha-halogenated ketone having the structure

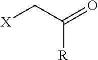

(vi) an hidrazide having the formula R—NH—NH—R,

(vii) nitric oxide and derivatives thereof containing the NO group,

(viii) cupric ions and complexes containing Cu²⁺,

(ix) ferric ions and complexes containing Fe³⁺,

wherein R is any atom or molecule and X is selected from the group consisting of F, I, Br and Cl.

Examples of disulfide compounds include, but are not limited to, the following: tetramethylthiuram disulfide, tetraethylthiuram disulfide, tetraisopropylthiuram disulfide, tetrabutylthiuram disulfide, dicyclopentamethylenethiuram disulfide, isopropylxanthic disulfide, O,O-diethyl dithiobis-(thioformate), benzoyl disulfide, benzoylmethyl disulfide, formamidine disulfide 2HCl, 2-(diethylamino)ethyl disulfide, aldrithiol-2, aldrithiol-4, 2,2-dithiobis(pyridine N-oxide), 6,6-dithiodinicotinic acid, 4-methyl-2-quinolyl disulfide, 2-quinolyl disulfide, 2,2-dithiobis(benzothiazole), 2,2-dithiobis(4-tert-butyl-1-Isopropyl)-imidazole, 4-(dimethylamino)phenyl disulfide, 2-acetamidophenyl disulfide, 2,3-dimethoxyphenyl disulfide, 4-acetamidophenyl disulfide, 2-(ethoxycarboxamido)phenyl disulfide, 3-nitrophenyl disulfide, 4-nitrophenyl disulfide, 2-aminophenyl disulfide, 2,2-dithiobis(benzonitrile), p-tolyl disulfoxide, 2,4,5-trichlorophenyl disulfide, 4-methylsulfonyl-2-nitrophenyl disulfide, 4-methylsulfonyl-2-nitrophenyl disulfide, 3,3-dithiodipropionic acid, N,N-diformyl-L-cystine, trans-1,2-dithiane-4,5-diol, 2-chloro-5-nitrophenyl disulfide, 2-amino-4-chlorophenyl disulfide, 5,5-dithiobis(2-nitrobenzoic acid), 2,2-dithiobis(1-naphtylamine), 2,4-dinitrophenyl-p-tolyl disulfide, 4-nitrophenyl-p-tolyl disulfide, and 4-chloro-3-nitrophenyl disulfideformamidine disulfide dihydrochloride.

In a preferred embodiment, the disulfide compound is selected from the group of disulfiram or aldrithiol-2 (2,2′-dithiodipyridine). In another preferred embodiment, the zinc finger-modifying compound is aldrithiol-2. In further preferred embodiment, the zinc finger-modifying compound is disulfiram.

An example of a maleimide is N-ethylmaleimide.

An example of a hydrazide is 2-(carbamoylthio)-acetic acid 2-phenylhydrazide.

In another embodiment, the inactivation is photochemical. In a preferred embodiment, the photochemical inactivation is carried out by using a psoralen compound and irradiating the mixture of the virus and the psoralen compound at a wavelength capable of activating said psoralen compound.

Psoralens may be used in the inactivation step include psoralen and substituted psoralens, in which the substituent may be alkyl, particularly having from one to three carbon atoms (e.g. methyl); alkoxy, particularly having from one to three carbon atoms (e.g. methoxy); and substituted alkyl having from one to six, more usually from one to three carbon atoms and from one to two heteroatoms, which may be oxy, particularly hydroxy or alkoxy having from one to three carbon atoms (e.g. hydroxy methyl and methoxy methyl), or amino, including mono- and dialkyl amino or aminoalkyl, having a total of from zero to six carbon atoms (e.g. aminomethyl). There will be from 1 to 5, usually from 2 to 4 substituents, which will normally be at the 4, 5, 8, 4′ and 5′ positions, particularly at the 4′ position.

Examples of psoralens include psoralen; 5-methoxypsoralen; 8-methoxy-psoralen; 5,8-dimethoxypsoralen; 3-carbethoxypsoralen ; 3-carbethoxy-pseudopsoralen; 8-hydroxypsoralen; pseudopsoralen; 4,5′,8-trimethyl-psoralen; allopsoralen; 3-aceto-allopsoralen; 4,7-dimethyl-allopsoralen; 4,7,4′-trimethyl-allopsoralen; 4,7,5′-trimethyl-allopsoralen; isopseudopsoralen; 3-acetoisopseudopsoralen; 4,5′-dimethyl-isopseudo-psoralen; 5′,7-dimethyl-isopseudopsoralen; pseudoisopsoralen; 3-aceto-seudoisopsoralen; 3/4′,5′-trimethyl-aza-psoralen; 4,4′,8-trimethyl-5′-amino-methylpsoralen; 4,4′,8-trimethyl-phthalamyl-psoralen; 4,5′,8-trimethyl-4′-aminomethyl psoralen; 4,5′,8-trimethyl-bromopsoralen; 5-nitro-8-methoxy-psoralen; 5′-acetyl-4,8-dimethyl-psoralen; 5′-aceto-8-methyl-psoralen; and 5′-aceto-4,8-dimethyl-psoralen. In a more preferred embodiment the psoralen compound is amotosalen, preferably in salt form as amotosalen hydrochloride (S-59). No in vivo pharmacological effect of residual amotosalen is intended.

The time of UV irradiation will vary depending upon the light intensity, the concentration of the psoralen, the concentration of the virus, and the manner of irradiation of the virus receives, where the intensity of the irradiation may vary in the medium. The time of irradiation will be inversely proportional to the light intensity. The total time will usually be at least about 5 minutes and no more than about 30 minutes, generally ranging from about 5 to 10 minutes.

The light, which is employed, will generally have a wavelength in the range from about 300 nm to 400 nm. Usually, an ultraviolet light source will be employed together with a filter for removing UVB light. The intensity will generally range from about 150 μW/cm² to about 1500 μW/cm², although in some cases, it may be higher.

It may be desirable to remove the unexpended psoralen or its by-products from the irradiation mixture. This can be readily accomplished by one of several standard laboratory procedures such as dialysis across an appropriately sized membrane or through an appropriately sized hollow fiber system after completion of the irradiation. Alternatively, affinity methods can be used for removing one or more of the low molecular weight materials.

3. Antigen-Loaded Dendritic Cells and Dendritic Cell Vaccines

The method according to the present invention allows obtaining antigen-pulsed dendritic cells. Thus, in another aspect, the invention relates to an antigen-pulsed dendritic cell which can be obtained by using the method according to the invention.

Dendritic cells suitable for this invention can be of different types such as, without limitation, myeloid DCs, plasmacytoid DCs, Langerhans cells and insterstitial DCs. The most potent of the professional APCs are DCs of myeloid origin. Thus, in a preferred embodiment, DCs are myeloid DCs.

Dendritic cells can be identified by their particular profile of cell surface markers. This determination can be carried out, for example, by means of flow cytometry using conventional methods and apparatuses. For example, a fluorescent-activated cell sorting (Becton Dickinson Calibur FACS, Becton-Dickinson Labware Inc., Franklin Lakes, N.J., US) system with commercially available antibodies following protocols well established in the art can be used. Thus, the cells presenting a signal for a specific cell surface marker in the flow cytometry above the background signal can be selected. The background signal is defined as the signal intensity given by a non-specific antibody of the same isotype as the specific antibody used to detect each surface marker in the conventional FACS analysis. In order for a marker to be considered positive, the observed specific signal has to be more than 20%, preferably, 30%, 40%, 50%, 60%, 70%, 80%, 90%, 500%, 1000%, 5000%, 10000% or above, intense in relation to the intensity of the background signal using conventional methods and apparatuses.

Dendritic cells have profound abilities to induce and coordinate T cell immunity. This makes them ideal biological agents for use in immunotherapeutic strategies to augment T cell immunity to HIV infection. Thus, in another embodiment, the invention relates to a vaccine comprising the antigen-pulsed dendritic cells which can be obtained using the method according to the invention.

Said dendritic cell vaccine is preferably autologous to the subject. As used herein, the term “autologous” is meant to refer to any material derived from the same subject to which it is later to be reintroduced into the subject. The most effective immunotherapeutic vaccines utilize antigen based on autologous HIV (i.e. the quasi-species of virus unique to each host). The most impressive results in anti-HIV immunotherapy trials to date have used dendritic cells loaded with whole, inactivated HIV virions derived from the subjects' autologous virus. The dendritic cells are also obtained from the same subject. In a preferred embodiment, the dendritic cell preparation is autologous to the subject from which the CD4+ T cells and the CD14+ monocytes have been isolated.

In another aspect, the invention relates to a dendritic cell vaccine according to the invention for use in medicine.

In another aspect, the invention relates to a dendritic cell vaccine according to the invention wherein the immunogen is an HIV immunogen for use in the treatment or prevention of an HIV-infection or a disease associated with an HIV infection.

In another aspect, the invention relates to the use of a dendritic cell vaccine according to the invention wherein the immunogen is an HIV immunogen for the preparation of a medicament for the treatment in a subject of an HIV-1 infection or a disease associated with an HIV infection.

In another aspect, the invention relates to a method of treatment of a subject afflicted with an HIV-1 infection or a disease associated with an HIV infection comprising the administration to said subject of a dendritic cell vaccine according to the invention wherein the immunogen is HIV.

The dendritic cell vaccine of the invention can be a therapeutic vaccine, that is, a material given to already HIV infected subjects that have developed AIDS to help fight the disease by modulating their immune responses. Therapeutic HIV vaccines represent promising strategy as an adjunct or alternative to current antiretroviral treatment options for HIV.

The dendritic cell vaccine of the invention can be a prophylactic AIDS vaccine designed to be administered to an already HIV infected subject that has not developed AIDS. The vaccine of the invention is not a prophylactic AIDS vaccine designed to prevent HIV infection of a healthy subject.

In a preferred embodiment, the dendritic cell vaccine of the invention is administered to a subject that is under antiretroviral therapy (ART), and preferably, under Highly Active Antiretroviral Therapy (HAART). In another preferred embodiment the dendritic cell vaccine of the invention is administered to a subject that has discontinued antiretroviral therapy.

Accordingly, the therapeutic vaccine finds application to reduce the replication of HIV-1 in already infected subjects and limit the infectivity of virus in a vaccinated subject.

Said dendritic cell vaccine can be an autologous dendritic cell vaccine. Thus, in a preferred embodiment the subject to be treated is the same subject from which the CD4+ T cells and the CD14+ monocytes were isolated.

The dendritic cell HIV therapeutic vaccine compositions are reinjected to the subject. Suitable routes of delivery of dendritic cell HIV therapeutic vaccines are intravenous, subcutaneous, intradermal or intranodal route. A combination of different routes is also possible.

The dendritic cell vaccine of the invention is an antigen-loaded dendritic cell preparation comprising an immunogenically effective amount of an essentially inactivated HIV according to the invention and a pharmaceutically acceptable carrier.

In another embodiment, the dendritic cell-based vaccines of the invention can be administered by, for example, direct delivery of the APC loaded with inactivated subtype-specific HIV (e.g. by a subcutaneous injector) to a subject by methods known in the art.

In another embodiment, an individual is treated with APCs loaded with inactivated HIV of a specific subtype. The APCs are first loaded with the inactivated HIV ex vivo. The loaded APCs are then administered to the subject by any suitable technique. Preferably, the loaded APCs are injected subcutaneously, intradermally or intramuscularly into the individual, preferably, by a subcutaneous injection. More preferably, the APCs are obtained by sampling PBMCs previously from the subject under treatment. The monocytes (CD 14+) isolated from the PBMCs are differentiated to immature dendritic cells which are then developed into mature dendritic cells. Such methods are well known in the art.

In another embodiment, the inactivated whole HIV is combined with an adjuvant to induce a cellular immune response against HIV-1. Suitable adjuvants include complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, conventional bacterial products (e.g. cholera toxin, heat-labile enterotoxin, attenuated or killed BCG (Bacillus Calmette-Guerin) and Corynebacterium parvum, or BCG derived proteins), biochemical molecules (e.g. TNF-α, IL-1-β, IL-6, PGE₂, or CD40L), or oligodeoxynucleotides containing a CpG motif. Examples of materials suitable for use in vaccine compositions have been disclosed previously. See Osol A, Ed., Remington's Pharmaceutical Sciences (Mack Publishing Co., Easton, Pa., US, 1980, pp. 1324-1341).

An adjuvant that may convene to the instant invention may be any ligand suitable for the activation of a pathogen recognition receptor (PRR) expressed in and on dendritic cells, T cells, B cells or other antigen presenting cells. Ligands activating the nucleotide-binding oligomerization domain (NOD) receptor pathway may be suited for the purpose of the invention. Adjuvants suitable for these ligands may be muramyl dipeptide derivatives. Ligands activating the toll-like receptors (TLRs) may also convene for the purpose of the invention. Those receptors are member of the PRR family and are widely expressed on a variety of innate immune cells, including DCs, macrophages, mast cells and neutrophils.

As example of ligands activating TLR, mention may be made, for TLR4 of monophosphoryl lipid A, 3-O-deacytylated monophosphoryl lipid A, LPS from E. coli, taxol, RSV fusion protein, and host heat shock proteins 60 and 70, for TLR2 of lipopeptides such as N-palmitoyl-S-2,3(bispalmitoyloxy)-propyl-cvsteinyl-seryl-(lysil)3-lysine, peptidoglycan of S. aureus, lipoproteins from M. tuberculosis, S. cerevisiae zymosan and highly purified P. gingivalis LPS; for TLR3 of dsRNA, TLR5 of flagellin and TLR7 synthetic compounds such as imidazoquinolines; or for TLR9 of certain types of CpG-rich DNA. Other useful adjuvants for the invention may be T helper epitopes.

The vaccines of the invention can be formulated into pharmaceutical compositions (also called “medicaments”) for treating an individual chronically infected with HIV. Pharmaceutical compositions of the invention are preferably sterile and pyrogen free, and also comprise a pharmaceutically acceptable carrier. Suitable pharmaceutically acceptable carriers include water, saline solutions (e.g. physiological saline), viscosity adjusters and other conventional pharmaceutical excipients or additives used in the formulation of pharmaceutical compositions for use in humans. Suitable pharmaceutical excipients include stabilizers, antioxidants, osmolality adjusting agents, buffers, and pH adjusting agents. Suitable additives include physiologically biocompatible buffers (e.g. tromethamine hydrochloride), chelants (e.g. DTPA, DTPA-bisamide) or calcium chelate complexes (e.g. calcium DTPA, CaNaDTP A-bisamide), or, optionally, additions of calcium or sodium salts (e.g. calcium chloride, calcium ascorbate, calcium gluconate, calcium lactate). The formulation of the pharmaceutical compositions of the invention is within the ability of a person with skill in the art. See Gennaro, 2003, supra.

A typical regimen for treating an individual chronically infected with HIV which can be alleviated by a cellular immune response by active therapy, comprises administration of an effective amount of a vaccine composition as described above, administered as a single treatment, repeatedly, with or without enhancing or booster dosages, over a period up to and including one week to about 24 months.

According to the present invention, an “immunogenically effective amount” of an essentially inactivated HIV or of an immunogenic composition of the invention is one which is sufficient to cause the subject to a specific and sufficient immunological response, so as to impart protection against subsequent HIV exposures to the subject. In this case, an effective amount causes a cellular or humoral response to HIV, preferably, a cellular immune response.

The immunogenically effective amount results in the amelioration of one or more symptoms of a viral disorder, or prevents the advancement of a viral disease, or causes the regression of the disease or decreases viral transmission. For example, an immunogenically effective amount refers preferably to the amount of a therapeutic agent that decreases the rate of transmission, decreases HIV viral load, or decreases the number of infected cells, by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more. An immunogenically effective amount, with reference to HIV, also refers to the amount of a therapeutic agent that increases CD4+ cell counts, increases time to progression to AIDS, or increases survival time by at least 5%, preferably, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more.

It is understood that the effective dosage will be dependent upon the age, sex, health, and weight of the recipient, kind of concurrent treatment, if any, frequency of treatment, and the nature of the effect desired. The most preferred dosage will be tailored to the individual subject, as is understood and determinable by one of skill in the art, without undue experimentation. See Gennaro, 2003, supra.

The efficacy of treatment of the invention can be assessed through different means such as, for example, by monitoring the viral load and CD4+ T cell count in the blood of an infected subject or by measuring cellular immunity.

The monitoring of the viral load and CD4+ T cell count in the blood is carried out by standard procedures. If the vaccine is efficacious, there should be greater than or equal to one log reduction in viral load, preferably to less than 10,000 copies/mL HIV-RNA within 2-4 weeks after the commencement of treatment. If a reduction in viral load of less than 0.5 log is attained, or HIV-RNA stays above 100,000, then the treatment should be adjusted by either adding or switching drugs. Viral load measurement should be repeated every 4-6 months if the subject is clinically stable. If viral load returns to 0.3-0.5 log of pre-treatment levels, then the therapy is no longer working and should be changed. Within 2-4 weeks of starting treatment, CD4+ T-cell count should be increased by at least 30 cells/mm³. If this is not achieved, then the therapy should be changed. The CD4+ T-cell counts should be monitored every 3-6 months during periods of clinical stability, and more frequently, should symptomatic disease occur. If CD4+ T-cell count drops to baseline (or below 50% of increase from pre-treatment), then the therapy should be changed.

To measure cellular immunity, cell suspensions of enriched CD4+ and CD8+ T cells from lymphoid tissues are used to quantify antigen-specific T cell responses by cytokine-specific ELISPOT assay. See Wu S, et al., 1995, 1997, supra. Such assays can measure the numbers of antigen-specific T cells that secrete IL-2, IL-4, IL-5, IL-6, IL-10 and IFN-γ. All ELISPOT assays are conducted using commercially-available capture and detection mAbs (R&D Systems, Inc., Minneapolis, Minn., USA; BD Biosciences Pharmingen, San Diego, Calif., USA). See Wu S, et al., 1995, 1997, supra; Shata M, 2001, supra. Each assay includes mitogen (Con A) and ovalbumin controls.

In the context of the present invention “HIV antigen” is the whole inactivated HIV virus which is capable of generating an immune response in a subject. Said immune response can be the production of antibodies or cell-mediated immune responses against the virus.

Particularly, “immune response” refers to a CD8+ T cell mediated immune response to HIV infection. An immune response to HIV may be assayed by measuring anyone of several parameters, such as viral load, T cell proliferation, T cell survival, cytokine secretion by T cells, or an increase in the production of antigen-specific antibodies (e.g. antibody concentration).

Thus, the immunogenic compositions of the invention are useful for preventing HIV infection or slowing progression to AIDS in infected individuals. The compositions containing HIV antigen produced from HIV grown in chemically defined, protein free medium and methods of using such compositions can be used to elicit potent Th1 cellular and humoral immune responses specific for conserved HIV epitopes, elicit HIV-specific CD4 T helper cells, HIV-specific cytotoxic T lymphocyte activity, stimulate production of chemokines and cytokines such as β-chemokines, IFN-γ, interleukin 2 (IL-2), interleukin 7 (IL-7), interleukin 15 (IL-15), or α-defensin, and increase memory cells. Such vaccines can be administered via various routes of administration. Such vaccines can be used to prevent maternal transmission of HIV, for vaccination of newborns, children and high-risk individuals, and for vaccination of infected individuals. Such vaccines can optionally include immunomers or an immunostimulatory sequence (ISS) to enhance an immune response against the HIV antigen. Such vaccines can also be used in combination with other HIV therapies, including antiretroviral therapy with various combinations of nuclease and protease inhibitors and agents to block viral entry, such as T20. See Baldwin C, et al., Curr. Med. Chem. 2003; 10:1633-1642.

The immunogenic compositions of the invention when administered to a subject that has no clinical signs of the infection can have a preventive activity, since they can prevent the onset of the disease.

The beneficial prophylactic or therapeutic effect of an HIV immunogenic composition in relation to HIV infection or AIDS symptoms include, for example, preventing or delaying initial infection of an individual exposed to HIV; reducing viral burden in an individual infected with HIV; prolonging the asymptomatic phase of HIV infection; maintaining low viral loads in HIV infected subjects whose virus levels have been lowered via anti-retroviral therapy; increasing levels of CD4 T cells or lessening the decrease in CD4 T cells, both HIV-1 specific and non-specific, in drug naive subjects and in subjects treated with ART, increasing overall health or quality of life in an individual with AIDS; and prolonging life expectancy of an individual with AIDS. A clinician can compare the effect of immunization with the subject's condition prior to treatment, or with the expected condition of an untreated subject, to determine whether the treatment is effective in inhibiting AIDS.

In a preferred embodiment, the immunogenic compositions of the invention are preventive compositions.

The immunogenic compositions of the invention may be useful for the therapy of HIV-1 infection. While all animals that can be afflicted with HIV-1 or their equivalents can be treated in this manner (e.g. chimpanzees, macaques, baboons or humans), the immunogenic compositions of the invention are directed particularly to their therapeutic uses in humans. Often, more than one administration may be required to bring about the desired therapeutic effect; the exact protocol (dosage and frequency) can be established by standard clinical procedures.

All publications mentioned hereinabove are hereby incorporated in their entirety by reference.

While the foregoing invention has been described in some detail for purposes of clarity and understanding, it will be appreciated by one skilled in the art from a reading of this disclosure that various changes in form and detail can be made without departing from the true scope of the invention and appended claims.

### EXAMPLES

### General Procedures 1. Isolation and Expansion of Autologous HIV

Fresh blood was extracted from an HIV positive donor subject and stored in a tube with ACD (acid citrate dextrose), CPD (citrate phosphate dextrose) or EDTA (ethylenediaminetetraacetic acid) as anticoagulant. Then, peripheral blood mononuclear cells (PBMCs) were separated from the blood by a Ficoll density gradient (Accuspin Histopaque®, Sigma-Aldrich Corp., Saint Louis, Mo., US). CD14+ monocytes were selected from the PBMCs by a CD14 antibody magnetic microbead system (CliniMACS® CD14 Microbeads, Miltenyi Biotech GmbH, Bergisch Gladbach, Del.) according to the manufacturer's procedure. Next, CD4+ T cells were isolated from the remaining solution (PBMC-CD14(−)) by a CD4+ antibody magnetic microbead system (CliniMACS® CD4 Microbeads, Miltenyi Biotech GmbH, Bergisch Gladbach, Del.) according to the manufacturer's procedure. Finally, the CD14+ monocytes and CD4+ T cells were suspended in X-VIVO15 serum free hematopoietic cell medium (BioWhittaker Inc., Walkersville, Md., US) supplemented with 10% human AB serum.

2. CD4+ T Cells and MΦ Co-Culture

The CD4+ T cells of the previous step were activated with CD3 (Orthoclone OKT3®, Janssen-Cilag, Johnson & Johnson, New Brunswick, N.J., US) and IL-2 (18.00 IU×10⁶, Proleukin®, Prometheus Labs., San Diego, Calif., US). The CD14+ monocytes were differentiated into macrophages. Afterwards, the CD4+ T cells and macrophages were co-cultured.

The method of activation of CD4+ T cells with CD3 started between 5 and 7 days before the co-culture of CD4+ T cells and macrophages is started. First, culture flasks were pretreated with a solution of 5 μg/mL CD3 in DPBS (i.e. Dulbecco's phosphate buffered saline) and incubated at 37° C. in horizontal position during at least 2 hours to allow that CD3 antibodies adhere to the flask wall. Then, the solution was discarded and the flask was washed two times with DPBS. After that, CD4+ T cells obtained from PBMCs were resuspended in an ex vivo activation culture medium composed by X-VIVO 15 medium supplemented with 10% human AB serum and 100 U/mL IL-2. Said suspension was incubated in the flask previously activated with CD3 in horizontal position at 37° C. and 5% CO₂ for about 24-48 hours.

Five days before the start of the co-culture, the activation of CD4+ T cells with CD3 was finished and fresh IL-2 was added. Briefly, the pre-activated CD4+ T cells were resuspended in the culture medium and washed two times with DPBS. After that, cells were resuspended in activation medium lacking CD3 (X-VIVO 15+10% human AB serum+100 U/mL IL-2) at 10⁶ cells/mL and incubated in a flask in vertical position at 37° C. and 5% CO₂ between 3 and 5 additional days to complete the proliferation of CD4+ T cells.

The differentiation of CD14+ monocytes to macrophages also started between 5 and 7 days before the co-culture of CD4+ T cells and macrophages. CD14+ monocytes isolated from PBMCs were resuspended in ex vivo culture media composed by X-VIVO15 medium and supplemented with 10% human AB serum. The suspension was incubated in an ULA flask (Corning®, Cultek SUL, Barcelona, ES) at 37° C. and 5% CO₂ in vertical position during 5-7 days to obtain mature macrophages.

The co-culture of CD4+ T cells and macrophages started between 5 and 7 days after the blood extraction. CD4+ T cells and macrophages were co-cultured in the ULA flask where the differentiation of CD14+ monocytes took place. The co-culture started with a relation of CD4+ T cells:macrophages of 1:1 and a density of 10⁶ cells/mL in a culture medium composed of X-VIVO15 medium supplemented with 10% human AB serum. When the number of CD4+ T cells is low and it is not possible to make a co-culture 1:1 (CD4+ T cells:macrophages), the co-culture can be 1:10 or 1:100 (CD4+ T cells:macrophages) by adjusting the medium to reach a cell density in the co-culture of 10⁶ cells/mL. When necessary, IL-2 was added to obtain a final concentration in the co-culture of 100 IU/mL IL-2. The flask was incubated in vertical position at 37° C. and 5% CO₂ during 7-60 days, preferably 7-21 days. The co-culture of CD4+ T cells and macrophages for the isolation and production of virus has a minimum length of 7 days and could be extended to 48-60 days. Once the co-culture is established, the medium has to be changed every 7 days.

The viral culture was monitored to analyze the production of virus during the co-culture by testing supernatants for both HIV-1 p24 antigen production/mL of supernatant by ELISA (Ag HIV®, Innogenetics NV, Ghent, BvE) and for HIV-1 RNA copy number/mL of supernatant by real time RT-PCR (PCR Real Time COBAS TAQMAN HIV-1 Test, v1.5, Roche Diagnostics Inc., Indianapolis, Ind., US) at days 7, 14, 21 and so for of the cell co-culture.

With this method it is possible to produce micrograms of HIV-1 p24 antigen/mL of supernatant at 7 days from HIV-1 positive subjects having >500 CD4 and 4 log copies of HIV-1 RNA/mL of plasma.

3. HIV Heat Inactivation

HIV contained in the supernatant from the step 2 was heat inactivated following protocols published previously to yield a lysate of essentially inactivated HIV. See Gil, 2011, supra.

a) Donor Subjects Off cART

Several 10 mL aliquots of a CD4+ T cells and MΦ co-culture supernatant containing HIV were inactivated by heat-treatment at 56° C. with agitation using a thermomixer (model AG 22331, Eppendorf AG, Hamburg, Del.) at 750 rpm for 30 min. The heat inactivated supernatants were concentrated by ultrafiltration using sterile centrifugal filter units (VivaSpin 20, 300 kDa, model VS2051, Sartorius AG, Gottingen, Del.) at 6000×g for 60 min at 21° C. For each donor subject, multiple VivaSpin 20 filters were needed to concentrate the total pooled supernatant volume of approximately 80 mL. Centrifugal filtration concentrates were washed using physiological saline solution (three times 6000×g for 60 min at 21° C.). The 0.5 mL final volume recovered from each centrifugal filter was pooled and centrifuged at 15,000×g (CH 007466 rotor and Heraeus Multifuge 1LR, Thermo Fisher Scientific Inc., Waltham, Mass., US) for 2 h at 4° C. The pellets were resuspended and pooled into a 1 mL of physiological saline solution and divided into 5 aliquots of immunogen of 0.2 mL each. The solutions were stored frozen at −80° C. until usage.

b) Donor Subjects On cART

Several 10 mL aliquots of a CD4+ T cells and MΦ co-culture supernatant containing HIV were inactivated by heat-treatment at 56° C. with agitation using a thermomixer (model AG 22331, Eppendorf AG, Hamburg, Del.) at 750 rpm for 30 min. The heat inactivated supernatants were concentrated by ultracentrifugation instead of ultrafiltration, as in the stage before. The supernatants were concentrated by ultracentrifugation at 100,000×g for 32 min at 4° C. in sterile polyallomer bottles (model 55083, Seton Scientific Inc., Petaluma, Calif., US) using a T1250 fiberlite rotor followed by another ultracentrifugation at 192,000×g for 10 min at 4° C. and in sterile 1.5 mL tubes (model 357448, Beckman Coulter Inc., Brea, Calif., US) using a F45L-24X1.5 fiberlite rotor and a Sorvall WX Ultra 80 centrifuge (Thermo Fisher Scientific Inc., Waltham, Mass., US). Final pellets were pooled into 1 mL of physiological saline and divided into 5 aliquots of immunogen of 0.2 mL each. The solutions were stored frozen at −80° C. until use.

4. HIV Chemical Inactivation

HIV contained in the supernatant from the step 2 was inactivated with a chemical agent according to procedures known in the art to yield a lysate of essentially inactivated HIV. See EP 11382358.7 filed on Nov. 22, 2011. The following chemical agents were utilized:

a) Aldrithiol-2 (2,2′-dithiodipyridine)

10 mL of a CD4+ T cells and MΦ co-culture supernatant containing HIV was treated with aldrithiol-2 (2,2′-dithiodipyridine) (AT-2, Aldrithiol-2®, catalogue number 143049, Sigma-Aldrich Corp., Saint Louis, Mo., US) according to protocols known in the art. See Rossio J, et al., J. Virol. 1998; 72(10):7992-8001 and Arthur L, et al., AIDS Res. Hum. Retroviruses 1998; Suppl 3:S311-S319. The supernatant could be incubated with AT-2 1 mM at 37° C. for 2 h under continuous agitation or, alternatively, at 4° C. for 24 h.

b) Disulfiram

10 mL of a CD4+ T cells and MΦ co-culture supernatant containing HIV was treated with disulfiram (Antabuse®, Odyssey Pharmaceuticals Inc., East Hanover, N.J., US) according to Chertova E., et al., Preparation of inactivated autologous subject derived HIV-1 for therapeutic vaccination, HIV Immunobiology: From Infection to Immune Control (X4) 2009, Keystone, Colo., US. The supernatant was incubated with disulfiram 0.3 mM at 37° C. for 3 h.

c) Azodicarbonamide

A first amount of azodicarbonamide (ADA) (HPH116, CAS number 123-77-3) was added to the supernatant containing HIV obtained from the previous step to inactivate the virus and incubated for 2 hours at 37° C. This inactivation was further reinforced by the addition of a second amount of azodicarbonamide to the solution. The solution was incubated for 2 hours to complete a total time of incubation of 4 hours. Then, the solution was centrifuged to obtain a first pellet. The first pellet was dissolved in physiological saline solution. The resulting solution was ultracentrifuged to obtain a second pellet. The second pellet was then dissolved in physiological saline solution to obtain a concentrate of the inactivated HIV.

d) Amotosalen

A first amount of amotosalen (AMT HCl, CAS number 161262-45-9, INTERCEPT®, Cerus Corp., Concord, Calif., US) was added to the supernatant containing HIV obtained from the previous step and incubated during 30 minutes. The solution was treated with ultraviolet radiation to inactivate the virus. Then, the solution was ultracentrifuged to obtain a first pellet. The first pellet was dissolved in physiological saline solution. The resulting solution was centrifuged to obtain a second pellet. The second pellet was then dissolved in physiological saline solution to obtain a concentrate of the inactivated HIV.

5. Quality Control

To calculate the median tissue culture infective dose (TCID50) of a viral stock and to quantify the infectivity reduction produced by the inactivation method and the residual infectivity that remains in the sample after the inactivation method, an assay to titrate HIV was done in PBMC.

First, fresh blood was obtained from a healthy donor and stored in a tube with ACD (acid citrate dextrose), CPD (citrate phosphate dextrose), EDTA (ethylenediaminetetraacetic acid) or heparin as anticoagulant. PBMCs were separated from the blood by a Ficoll density gradient three days before starting the titration. HIV-1, HBsAg and HCV antibodies as well as HCV PCR were negative. Then, PBMCs were activated with phytohaemagglutinin phosphate PHA-P (Sigma-Aldrich Corp., Saint Louis, Mo., US) by incubating cells in RPMI basic medium (RPMI 1640+20% fetal bovine serum+antibiotics) with phytohaemagglutinin 5 μg/mL during 1-3 days at 37° C. in a CO₂ incubator.

After that, the cells previously stimulated with phytohaemagglutinin were resuspended in viral culture medium (RPMI 1640+10 IU/mL IL-2+20% fetal bovine serum+antibiotics). 200 μl of inactivated autologous HIV-1, concentrated and diluted in physiological saline solution to a dilution 1/15 were analyzed. Said dilution is equivalent to the dilution that will be used to pulse dendritic cells. Additionally, 200 μl of autologous HIV-1 not inactivated and not concentrated was analyzed in viral culture medium (RPMI 1640+10 IU/mL IL-2+20% fetal bovine serum+antibiotics). Cells were incubated with viral dilutions overnight at 37° C. with CO₂.

The inactivation method with amotosalen, disulfiram, aldrithiol-2 or azodicarbonamide does not affect the conformation of the p24 protein. Thus, after the infection, the cells inoculated with the virus were washed to discard the excess of p24 protein in the supernatant and distinguish it from the new product produced after the infection. Then, cells resuspended in viral culture medium were incubated for 10-11 additional days at 37° C. with CO₂. The culture medium was changed at day 5 or 6.

The antigen p24 was determined by ELISA (HIV-1 p24 antigen-ELISA, catalogue number K1048, Innogenetics NV, Gent, BE). The criteria to conclude if the supernatant sample is positive or negative is based on the results of the standardized controls of HIV p24 antigen from the kit used for detecting the p24 antigen, that has an average sensitivity of 22 pg/mL.

Thus, the supernatant of the culture was considered qualitatively positive when [(OD Ag p24 in the problem well)-(OD Ag p24 in the control well p24 background)] was superior to the OD corresponding to the control of 22 pg/mL. And the supernatant of the culture was considered qualitatively negative when [(OD Ag p24 in the problem well)-(OD Ag p24 in the control well p24 background)] was inferior or equal to the OD corresponding to the control of 22 pg/mL. OD: optical density.

The TCID50 was calculated according to the Spearman-Kärber formula:

M=xk+d[0.5−(1/n)(r)]

wherein,

xk=dose of highest dilution

r=sum of negative responses

d=spacing between dilutions

n=number of wells per dilution

Then, the TCID50 value was corrected by the concentration factor (CF). See Karber G, Arch. Exper. Pathol. Pharmakol. 1931; 162:480-483 and Spearman C, Br. J. Psychol. 1908; 2:227-242.

### Example 1

### Ex Vivo Generation of Monocyte-Derived Dendritic Cells (MDDCs)

150 mL of fresh blood was extracted from a donor subject with HIV. Then, peripheral blood mononuclear cells (PBMCs) were separated from the blood by a Ficoll density gradient (Accuspin Histopaque®, Sigma-Aldrich Corp., Saint Louis, Mo., US). The resulting solution was centrifuged at 1200 rpm for 5 minutes.

The suspension was separated into 18 mL aliquots. The aliquots were poured into 75 cm² adhesive culture flasks (Corning Inc., Corning, N.Y., US) in horizontal position and placed in an incubator at 37° C. with humidified 5% CO₂ atmosphere for 2-3 hours. The non-adhered cells (lymphocytes) were isolated by suction. The adhered cells were mostly monocytes.

The adhered cells (monocyte layer) were washed 4 times with 15 mL of X-VIVO10 (cGMP, Biowhittaker Inc., Walkersville Md., US) pre-heated at 37° C. The solution was stirred carefully to eliminate possible lymphocyte contaminants deposited by gravity without removing the adhered monocytes.

Subsequently, the solution supernatant was discarded. The cells were re-suspended in a medium (“basic culture medium”) composed of X-VIVO15 (cGMP, Biowhittaker Inc., Walkersville Md., US) supplemented with 1% autologous inactivated serum, gentamicine (50 μg/mL, catalogue number 636183, B. Braun Medical S.A., Barcelona, ES), fungizone (2.5 μg/mL, catalogue number 760645, Bristol-Myers Squibb SL, Elche, ES) and AZT (1 μM, Retrovir®, GlaxoSmithKline plc, London, GB) at a concentration of 3-4×10⁶ cells/mL.

The adhered monocytes were cultured in the same flasks for 5 days. 18 mL of a medium (“basic culture medium”) composed of X-VIVO15 (cGMP, Biowhittaker Inc., Walkersville Md., US) supplemented with 1% autologous inactivated serum, gentamicine (50 μg/mL, catalogue number 636183, B. Braun Medical S.A., Barcelona, ES), fungizone (2.5 μg/mL, catalogue number 760645, Bristol-Myers Squibb SL, Elche, ES) and AZT (1 μM, Retrovir®, GlaxoSmithKline plc, London, GB). 1000 IU/mL IL-4 and 1000 IU/mL recombinant human (rh) GM-CSF (cGMP quality CellGenix GmbH, Freiburg, Del.) were added as well to each flask. IL-4 and GM-CSF were added to the culture every 2 days at the same concentrations.

After 5 days of culture, MDDCs were collected by washing the flasks 4 times with 15 mL of X-VIVO10 to favor the removal of MDDCs still adhered to the bottom. The MDDCs were collected in 50 mL tubes and were washed 2 times by centrifugation (2000 rpm for 5 minutes) with 50 mL of X-VIVO10. The MDDC pellet was re-suspended in 10 mL of X-VIVO10 and stored at 4° C. until use. A 200 μl aliquot was separated for quality control.

### Example 2

### Autologous MDDC Maturation and Pulsing with Inactivated HIV-1 in Adherent Surface Flasks

After 5 days of culture, 10.5 million MDDCs obtained as in Example 1 were centrifuged at 2000 rpm for 5 minutes. The sediment was resuspended in 2.8 mL of basic medium. See Example 1. A 0.2 mL aliquot of inactivated HIV-containing >10⁸ copies of HIV-1 RNA which has been previously resuspended with X-VIVO15 medium were added. The cells were plated on 75 cm² culture flasks with adherent surface in a vertical and slightly inclined position. 1000 IU/mL IL-4 and 1000 IU/mL recombinant human (rh) GM-CSF (cGMP quality CellGenix GmbH, Freiburg, Del.) were added to each flask and the cells incubated at 37° C.

After the incubation, 22 mL of basic culture medium were added together with GM-CSF and IL-4 at 1000 IU/mL and a maturation cocktail with the cytokines IL-6, TNF-α and IL-1-β at 1000 IU, 1000 IU and 300 IU/mL (cGMP quality, CellGenix GmbH, Freiburg, Del.), respectively. The cells were cultured in said medium for additional 44 hours.

After 48 h of culture, an aliquot of the pulsed MDDCs was retrieved for quality control, which included: counting of viable mature cells, determination of the percentage of viability, immunophenotyping and microbiological control by means of Gram staining.

The cells were washed three times in clinical saline solution supplemented with 1% pharmaceutical human albumin by sequential cycles of centrifugation at 2000 rpm for 5 minutes and resuspension of the cell pellet in the clinical saline solution. The cells were resuspended in 0.5 mL of said solution.

### Example 3

### Autologous MDDC Maturation and Pulsing with Inactivated HIV-1 in Ultra Low Attachment Flasks

After 5 days of culture, 10.5 millions MDDCs were centrifuged at 2000 rpm for 5 minutes. The sediment was resuspended in 2.8 mL of basic medium. See Example 1. A 0.2 mL aliquot of inactivated HIV-containing >10⁸ copies of HIV-1 RNA which has been previously resuspended with X-VIVO15 medium were added. The cells were plated on 75 cm² Ultra Low Attachment Surface culture flasks (Corning®, catalogue number 153814, Cultek, SLU, Madrid, ES) in a vertical and slightly inclined position. 1000 IU/mL IL-4 and 1000 IU/mL recombinant human (rh) GM-CSF (cGMP quality, CellGenix GmbH, Freiburg, Del.) were added to each flask and the cells incubated at 37° C. for 2-4 h with the flask in a slightly inclined position.

After the incubation, 22 mL of basic culture medium were added together with GM-CSF and IL-4 at 1000 IU/mL and a maturation cocktail with the cytokines IL-6, TNF-α, and IL-1-β at a concentration of 1000 IU, 1000 IU, and 300 IU per mL, respectively The cells were cultured in said medium for 44 additional hours in horizontal position.

After 48 h of culture, an aliquot of the pulsed MDDCs was retrieved quality control, which included: counting of viable mature cells, determination of the percentage of viability, immunophenotyping and microbiological control by means of Gram staining.

The cells were washed three times in clinical saline solution supplemented with 1% pharmaceutical human albumin (Grifols, S A, Barcelona, ES) by sequential cycles of centrifugation at 2000 rpm for 5 minutes and resuspension of the cell pellet in the clinical saline solution. The cells were resuspended in 0.5 mL of said solution.

### Example 4

### Effect of PGE₂ and the Use of Ultra Low Attachment Flasks on the Maturation of MDDCs

After 5 days of culture, 10.5 million MDDCs were centrifuged at 2000 rpm for 5 minutes. The sediment was resuspended in 2.8 mL of basic medium. See Example 1. A 0.2 mL aliquot of inactivated HIV-containing >10⁸ copies of HIV-1 RNA which has been previously resuspended with X-VIVO15 medium were added. The cells were plated on 75 cm² Ultra Low Attachment Surface culture flasks (Corning®, catalogue number 153814, Cultek, SLU, Madrid, ES) in a vertical and slightly inclined position. 1000 IU/mL IL-4 and 1000 IU/mL recombinant human (rh) GM-CSF (cGMP quality, CellGenix GmbH, Freiburg, Del.) were added to each flask and the cells incubated at 37° C. for 2-4 h with the flask in a slightly inclined position.

After the incubation, 22 mL of basic culture medium were added together with GM-CSF and IL-4 at 1000 IU/mL and a maturation cocktail with the cytokines IL-6, TNF-α, IL-1-β, and PGE₂ at a concentration of 1000 IU, 1000 IU, 300 IU, and 1 μg per mL, respectively. The cells were cultured in said medium for 44 additional hours in horizontal position.

After 48 h of culture, an aliquot of the pulsed MDDCs was retrieved quality control, which included: counting of viable mature cells, determination of the percentage of viability, immunophenotyping and microbiological control by means of Gram staining.

The cells were washed three times in clinical saline solution supplemented with 1% pharmaceutical human albumin (Grifols, S A, Barcelona, ES) by sequential cycles of centrifugation at 2000 rpm for 5 minutes and resuspension of the cell pellet in the clinical saline solution. The cells were resuspended in 0.5 mL of said solution.

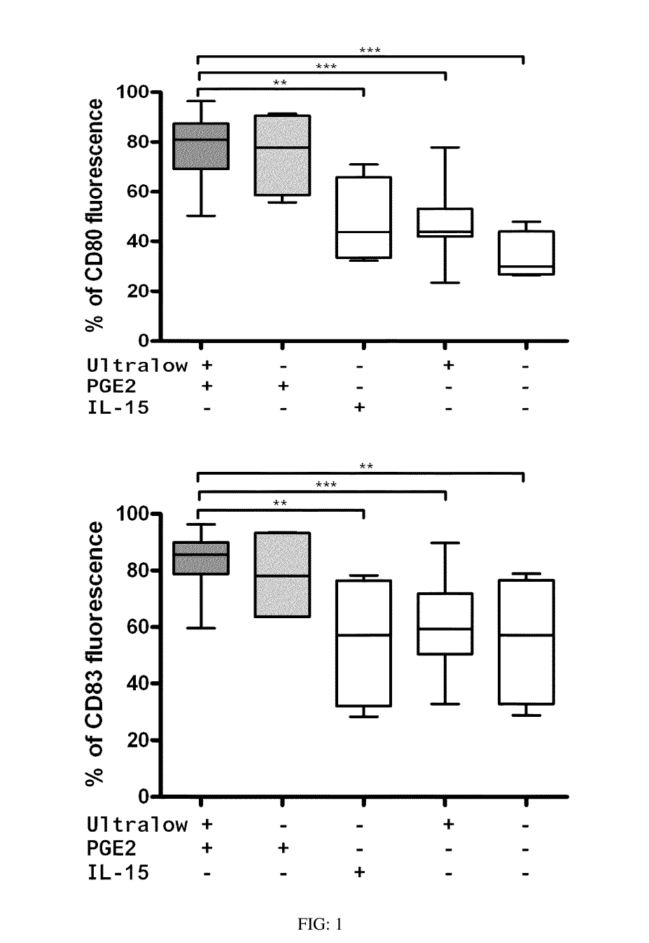

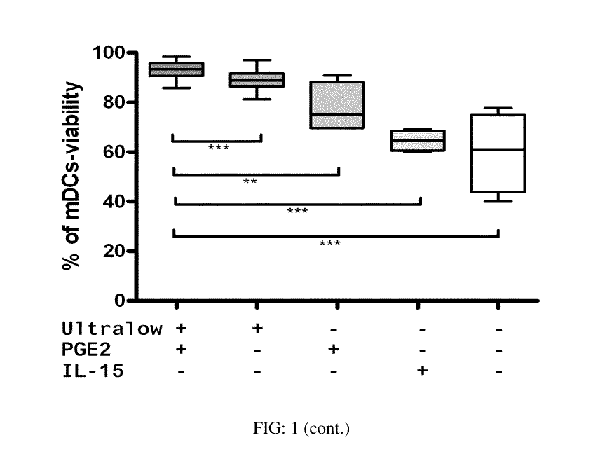

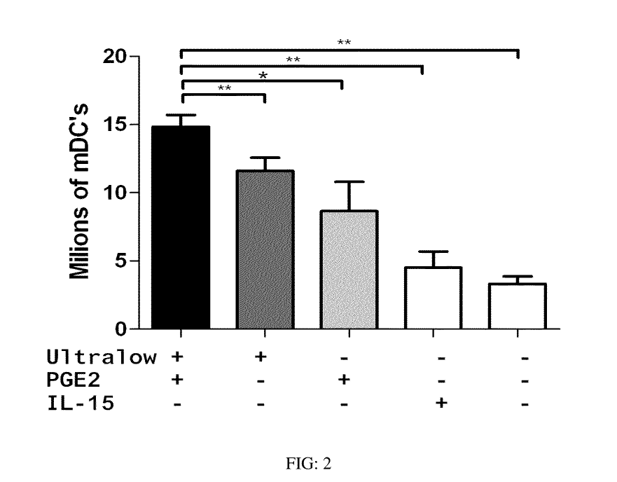

Experiments were conducted to assess if the addition of PGE₂ to the maturation cocktail increased the maturation markers CD80 and CD83 in the MDDCs of HIV positive subjects (with or without PGE₂, Ultralow attachment flasks and IL-15). See FIG. 1. The cells were analyzed by flow cytometry after maturation. The fluorescence intensity of markers CD80 and CD83 was assessed with antibodies specific against said clusters. The maturation with a cocktail of cytokines and PGE₂ induced a greater quantity of CD80 (nearly 2-folds higher) and CD83 (nearly 1.5-folds higher) markers, compared to when PGE₂ was absent. The use of anti-adherent flasks in combination with the addition of PGE₂ to the maturation cocktail improved considerably the quality of the end product (mDCs) in terms of maturation, viability, yield and overall immunogenic potency, since a higher number of pulsed viable cells is associated with a greater immune response. See FIGS. 1 and 2. Remarkably, the use of anti-adherent flasks (Ultralow attachment flasks) increased 3-fold the amount of mDCs obtained. By applying the Mann-Whitney non-parametric statistics function test, significant differences between several methods (with or without ultralow flasks, PGE₂, and IL-15) versus the phase II method (p<0.05) were observed. See FIG. 2.

MDDCs must express the CCR7 receptor, among others, to enable their migration to the lymph nodes after maturation. This is accomplished through the action of several cytokines (i.e. CCL19, CCL21) which bind to the CCR7 receptor and attract MDDCs to the lymph nodes.

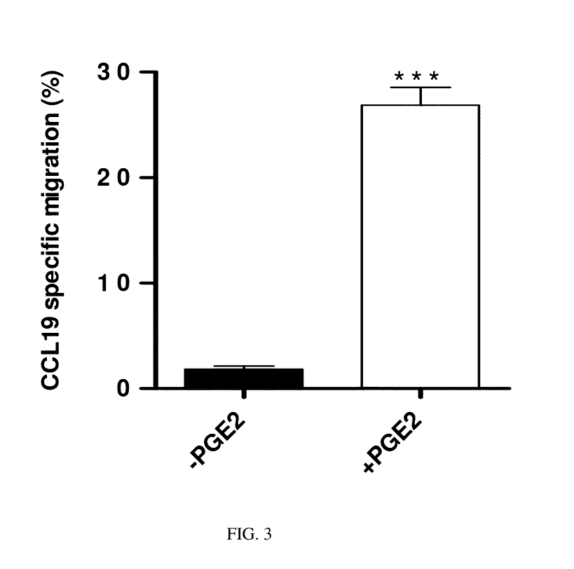

To assess the migration capacity of matured MDDCs obtained with or without PGE₂, an ex vivo migration assay was performed in transwell plates (Corning Inc., Corning, N.Y., US). Briefly, the MDDCs of 4 HIV positive subjects were induced to maturation with a cocktail of cytokines with or without PGE₂. The MDDCs (50,000 per well) were deposited in the upper compartment of the wells. The MDDC culture medium was used as negative control, while CCL19 in the MDDC culture medium was used to assess specific migration. The CCL19 chemokine was deposited in the lower compartment and was separated from the upper compartment by a 5 μm membrane. After three hours of incubation, the MDDCs that migrated to the lower compartment were collected and quantified by flow cytometry (60 seconds). By applying the Student's T statistics function for unpaired data, significant differences between the two methods (p<0.005) were observed. CCL19-mediated migration was greater when a cocktail of cytokines with PGE₂ was used. See FIG. 3.

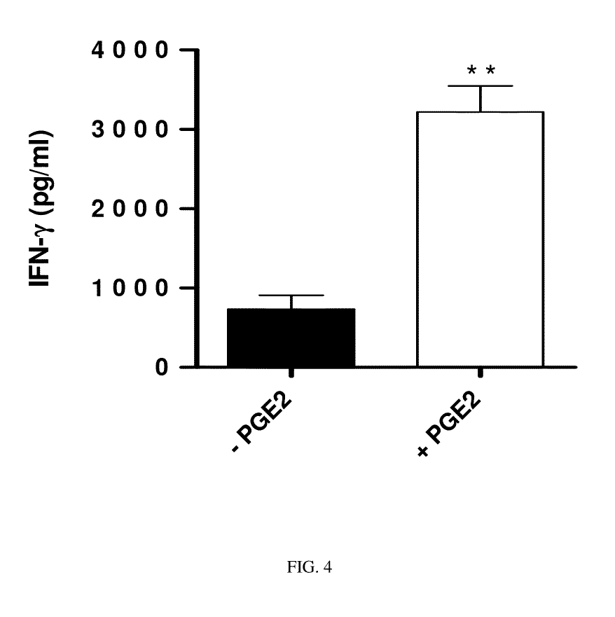

An additional experiment was conducted with MDDCs derived from HIV positive subjects with HIV specific T cell responses to evaluate if the matured MDDCs obtained by both methods were capable of promoting specific cellular responses. The MDDCs were pulsed with the HIV Bal virus. Then, they were induced to maturation with a cocktail of cytokines with or without PGE₂. After maturation, the cells were washed 4 times and put in contact with autologous T cells (from the same subject) in 96 well plates. The specific response against HIV was obtained by measuring the IFN-γ secreted by the lymphocytes in the supernatant of the MDDCz and lymphocyte co-cultures. By applying the Student's T statistics function for unpaired data, significant differences between the two methods (p<0.01) were observed. See FIG. 4. The MDDCs induced to maturation with the cocktail of cytokines plus PGE₂ elicited a greater specific response against HIV compared to without PGE₂.

### Example 5

### Vaccination with MDDCs Pulsed with Inactivated HIV-1 in Off cART Subjects

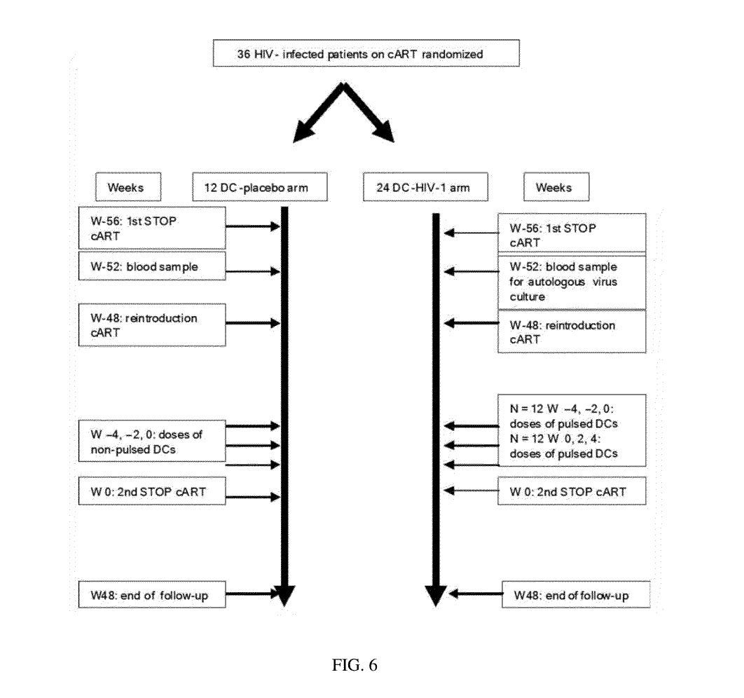

Thirty six subjects on successful cART and with CD4+>450 cells/mm³ were randomized to a blinded protocol (2:1) to receive: Arm 1 (cases or DC-HIV-1): immunizations every 2 weeks (a total of 3) with peripheral blood MDDCs (10⁷ cells) pulsed with ˜10⁹ virions of autologous inactivated HIV-1 (n=24); or Arm 2 (DC-placebo arm or DC-control): non-pulsed DCs (n=12). See FIG. 6. WO was considered the day of interruption of cART. The primary end-points were safety, change in viral load and proportion of subjects with a decrease in viral load ≧1 log₁₀ when compared to baseline before any cART vs week 12 and 24 after cART interruption. Secondary end-points were proportion of subjects required to restart cART as specified per protocol (drop of CD4 T cells below 300 cells/mm³ in at least 2 determinations separated by 15 days), changes in CD4 cell counts and in HIV-1 specific responses.

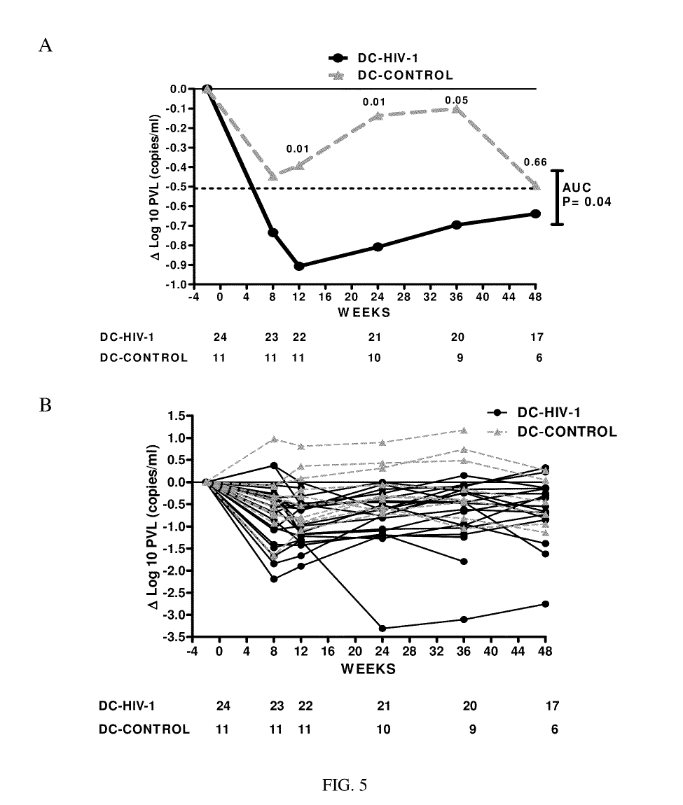

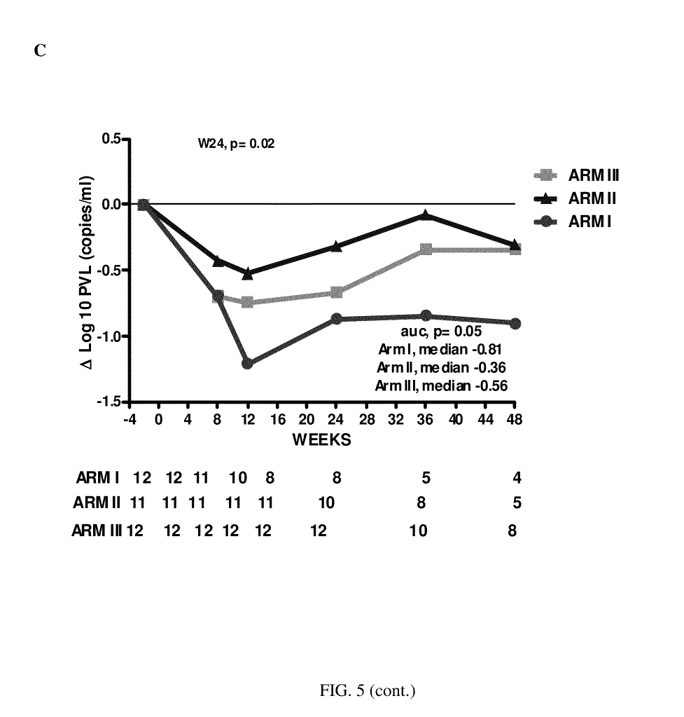

The dendritic cell vaccine was well tolerated, without any significant side effects. The mean decrease of viral load compared to pre-cART levels was −1.0 vs −0.46 log₁₀ at week 12 and −0.86 and −0.22 log₁₀ at week 24, in cases and controls, respectively (p=0.04 and p=0.03). At weeks 12 and 24, a decrease of viral load ≧1 log was observed in 10/22 (45%) vs 2/11(18%) and in 7/20 (35%) vs 0/10 (0%) in cases and controls, respectively (p=0.10, p=0.03). A significant difference in the change of viral load in the area-under-the-curve analysis was observed between immunized and control subjects (−0.72 vs −0.33, respectively, p=0.05). See FIG. 5C.

Results from example 5 have been reinterpreted by fusing the ARM 1 (DC-HIV-1, 12 subjects) and ARM 3 (DC-HIV-1, 12 subjects) curves. The curve ARM 2 remains the control (DC-control, 12 subjects). There are thirty six subjects in total. (see publication in www.ScienceTranslationalMedicine.org, 2 Jan. 2013, Vol 5 Issue 166 166ra, p. 1-9). See also FIG. 5A and 5B.

1-25. (canceled)

26. An in vitro method for obtaining antigen-loaded dendrite cells which comprises contacting immature dendritic cells with an immunogen comprising an antigen under conditions adequate for maturation of the immature dendri tie cells and under conditions which prevent the adhesion of the cells to a substrate.

27. The method according to claim 26, further comprising recovering the antigen-loaded dendritic cells.

28. The method according to claim 26, wherein the conditions adequate for maturation of the immature dendritic cells comprise contacting the immature dendrite cells with a combination of GM-CSF and IL-4.

29. The method according to claim 28, wherein the conditions adequate for maturation of the immature dendrite cells further comprise contacting the immature dendritic cells with a pro-inflammatory cytokine cocktail.

30. The method. according to claim 29, wherein the pro-inflainmatory cytokine cocktail comprises at least an agonist of the IL-1 receptor, a gp130 utilizing cytokine and a TN superfamily member.

31. The method according to claim 30, wherein the agonist of the IL-1 receptor is IL-1β, wherein the gp130 utilizing cytokine is IL-6 and/or wherein the TNF superfamily member is TNF-α.

32. The method according to claim 29, wherein the pro-inflammatory cytokine cocktail further comprises a prostaglandin.

33. The method according to claim 32, wherein the prostaglandin is prostaglandin E₂ (PGE₂).

34. The method according to claim 33, wherein the composition of the medium is 300 IU/mL of IL1β 1000 IU/mL of TNF-α, 1000 IU/mL of IL-6 and 1 μg/mL of PGE₂.

35. The method according to claim 26, wherein the conditions which prevent the adhesion of the cells to the substrate comprise the use of a low-adherence substrate.

36. The method according to claim 26, wherein the immature dendritic cells are monocyte-derived immature dendritic cells.

37. The method according to claim 26, wherein the immunogen is an HIV immunogen.

38. The method according to claim 37, wherein the HIV immunogen is an inactivated HIV particle.

39. The method according to claim 38, wherein the inactivated HIV particle is a selected from the group consisting of a heat-inactivated HIV particle, a chemically-inactivated HIV particle and a photochemically-inactivated HIV particle.

40. The method according to claim 39, wherein the chemically-inactivated HIV particle is obtained using an agent which disrupts CCHC zinc fingers, or wherein the photochemically-inactivated HIV is obtained using a psoralen compound and irradiation at a wavelength capable of activating the psoralen compound.

41. The method according to claim 40, wherein the agent which disrupts CCHC zinc fingers is selected from the group consisting of:
(i) a C-nitroso compound,
(ii) azodicarbonamide,
(iii) a disulphide having the structure R—S—S—R,
(iv) a maleimide having the structure 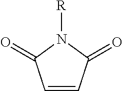
(v) an alpha-halogenated ketone having the structure 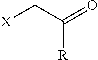
(vi) an hidrazide having the formula R—NH—NH—R,
(vii) nitric oxide and derivatives thereof containing the NO group,
(viii) cupric ions and complexes containing Cu²⁺, and
(ix) ferric ions and complexes containing
wherein R is any atom or molecule and X is selected from the group consisting of IF, I, Br and Cl.

42. The method according to claim 41, wherein the disulfide is disulfiram or aldrithiol-2 (2,2′-dithiodipyridine).

43. The method according to claim 40, wherein the psoralen compound is amotosalen.

44. The method according to claim 37, wherein the HIV is HIV-1.

45. An antigen-pulsed dendritic cell obtainable by a method which comprises contacting immature dendritic cells with an immunogen comprising the antigen under conditions adequate for maturation of the immature dendritic cells and under conditions which prevent the adhesion of the cells to a substrate.

46. A dendritic cell vaccine comprising an antigen-pulsed dendritic cell according to claim 45.

47. A method for the treatment or prevention of an HIV-infection or of a disease associated with an HIV infection in a subject in need thereof comprising administering to the subject a dendritic cell vaccine wherein the immunogen is an HIV immunogen and wherein the dendritic cell vaccine comprises an antigen-pulsed dendritic cell obtainable by a method which comprises contacting immature dendritic cells with an immunogen comprising the antigen under conditions adequate for maturation of the immature dendritic: cells and under conditions which prevent the adhesion of the cells to a substrate.
